(19) United States
(12) Patent Application Publication
(10) Pub. No.: US 2026/0151543 A1
(43) Pub. Date: Jun. 4, 2026

(54) DEVICES AND DELIVERY SYSTEM COMPONENTS CONTAINING SHAPE-MEMORY ALLOYS

(71) Applicant: Edwards Lifesciences Corporation, Irvine, CA (US)

(72) Inventors: Stephanie Chan O’keeffe, Tustin, CA (US); Dhiraj Catoor, Trabuco Canyon, CA (US); Nitin Sharma, Orange, CA (US); Jeremy Lawrence Valdecanas, Garden Grove, CA (US); Jeremy Schaffer, Fort Wayne, IN (US)

(21) Appl. No.: 19/459,941

(22) Filed: Jan. 26, 2026

Related U.S. Application Data
(63) Continuation-in-part of application No. PCT/US2025/051344, filed on Oct. 16, 2025.
(60) Provisional application No. 63/709,058, filed on Oct. 18, 2024.

(51) Int. Cl. A61L 31/02

(52) U.S. Cl. CPC A61L 31/022; A61L 2400/16; A61L 2400/18

(57) ABSTRACT

An implantable medical device is formed at least in part with a shape-memory alloy having a chemical composition of Ni₄₉₋₅₁Ti₂₈₋₃₈Hf₁₀₋₁₉Al_{0.1-3.5}. The implantable medical device has improved radiopaque properties compared with traditional shape memory alloys. The implantable medical device may be collapsible and re-expandable for facilitating advancement through the vasculature. The implantable medical device may be formed with a braided structure and/or foldable struts for enhancing collapsibility.

### RELATED APPLICATIONS

This application is a continuation in part of International Application No. PCT/US2025/051344, filed Oct. 16, 2025, which claims the benefit of U.S. Provisional Application No. 63/709,058, filed on Oct. 18, 2024, each of which is incorporated by reference herein.

### BACKGROUND

This application relates to implantable medical devices and delivery system components, and, more particularly, to radiopaque medical devices including nickel-titanium-hafnium-aluminum quaternary alloys.

Fluoroscopy and other X-ray-based visualization techniques are used to determine the anatomical location and orientation of implantable devices within a patient's body. X-ray-based visualization techniques aid in medical procedures, including, for example, implantation of stents, shunts, and replacement heart valves. In many instances, the superelastic materials necessary for implantable devices have inherently low radiopacity (low X-ray absorption). Traditionally, radiopaque markers are affixed to devices to aid visualization. However, the markers are typically small and provide only the marker's position rather than the entire body of the device to which they are attached. It is desirable to visualize the entire medical device to ensure proper placement and orientation within a patient.

### SUMMARY

Disclosed herein in one aspect is a device comprising: a shape memory alloy member formed at least partly from a superelastic shape-memory alloy, the alloy displaying reversible stress-induced martensite at a service temperature, such that it has a stress-induced martensitic state and an austenitic state, the shape memory alloy member having (i) a deformed shape when the alloy is in its stress-induced martensitic state and (ii) a different unstressed shape when the alloy is in its austenitic state; wherein the superelastic shape-memory alloy comprises, in atomic percent: a) nickel, b) titanium, c) a first element, wherein the first element is present in an amount effective to impart radiopaque properties to the memory alloy element; and d) a second element wherein the second element is present in an amount effective to balance an optional change in a martensite start temperature Mₛ and an austenite finish temperature A_{f} that occurs due to the presence of the first element and to reach a predetermined transformation temperature; wherein the superelastic shape-memory alloy is thermomechanically processed such that: (i) it exhibits superelastic properties at least at the service temperature, (ii) the alloy is substantially austenitic at zero applied stress at the service temperature, (iii) application of mechanical stress at the service temperature induces a martensitic phase transformation over a strain range, with austenite to martensite transformation stresses in a range of 500-2500 MPa, (iv) removal of the applied stress at the service temperature causes the stress-induced martensite to reversibly transform back to austenite without application of external heating; and wherein the shape memory alloy member exhibits a recoverable strain of 4 to 10% when deformed at the service temperature.

In one aspect also is disclosed a method comprising forming the shape memory alloy member of the device of any one of the examples herein from an ingot of a superelastic shape-memory alloy comprises, in atomic percent: a) nickel, b) titanium, c) a first element, wherein the first element is present in an amount effective to impart radiopaque properties to the memory alloy element; and d) a second element, wherein the second element is present an amount effective to balance an optional change in a martensite start temperature Mₛ and finish A_{f} that occurs due to the presence of Hf and to reach a predetermined transformation temperature.

In one aspect, disclosed is a method of manufacturing an implantable medical device or a delivery system component for an implantable medical device with radiopaque properties. In such aspects, the method can include converting a first ingot of a radiopaque quaternary alloy with a composition of Ni₄₉₋₅₁Ti₂₈₋₃₈Hf₁₀₋₁₉Al_{0.1-3.5} into a radiopaque quaternary alloy form, and forming the implantable medical device or the delivery system component from the radiopaque quaternary alloy form.

In another aspect, an implantable medical device with radiopaque properties can be formed from of a shape-memory alloy with chemical composition of Ni₄₉₋₅₁Ti₂₈₋₃₈Hf₁₀₋₁₉Al_{0.1-3.5}. The medical device may be balloon expandable, self-expanding, or mechanically expandable. The medical device may be formed with struts and/or may include a braided structure to assist with collapsibility, which is advantageous during delivery and implantation.

In another aspect, a delivery system component with radiopaque properties can include a braided layer for a sheath device comprised of a shape-memory alloy with a chemical composition of Ni₄₉₋₅₁Ti₂₈₋₃₈Hf₁₀₋₁₉Al_{0.1-3.5}. Yet in still further aspects, the braided layer can be referred to as a braided member.

In another aspect, a delivery system component with radiopaque properties can include a braided layer for a sheath device, comprised of a shape-memory alloy with a chemical composition of Ni₄₉₋₅₁Ti₂₈₋₃₈Hf₁₀₋₁₉Al_{0.1-3.5} alloy form, and forming the implantable medical device or the delivery system component from the radiopaque quaternary alloy form.

### BRIEF DESCRIPTION OF THE DRAWINGS

FIG. 1 is a schematic cross section of an implantable medical device.

FIG. 2 is a first flow chart showing a method of using wrought methods to create a wire that can be used to make the implantable medical device.

FIG. 3 is a second flow chart showing a method of using wrought methods to create a tube that can be used to make the implantable medical device.

FIG. 4A is a first fluoroscopic image comparing the radiopacity of different alloy compositions at a first thickness.

FIG. 4B is a second fluoroscopic image comparing the radiopacity of different alloy compositions at the first thickness.

FIG. 5A is a first fluoroscopic image comparing the radiopacity of different alloy compositions at a second thickness.

FIG. 5B is a second fluoroscopic image comparing the radiopacity of different alloy compositions at the second thickness.

FIG. 6 is a first graph showing engineering stress versus strain curves for different alloys.

FIG. 7 is a second graph showing engineering stress versus strain curves for different alloys.

FIG. 8 is a schematic diagram of a heart and vasculature.

FIG. 9 is a cross-sectional view of the heart.

FIG. 10A is a perspective view of a shunt device.

FIG. 10B is a side view of the shunt device.

FIG. 10C is a bottom view of the shunt device.

FIG. 11 is a perspective view of the shunt device in a collapsed configuration.

FIG. 12 is a side view of the shunt device with a sensor and anchored to a tissue wall.

FIG. 13A is a perspective view of a first example of a cardiovascular implant device.

FIG. 13B is a sectional view of a heart illustrating an example positioning at a non-valve site of the first example of the cardiovascular implant device.

FIG. 14A is a perspective view of a second example of a cardiovascular implant device.

FIG. 14B is a sectional view of a heart illustrating an example positioning at a valve site of the second example of the cardiovascular implant device.

FIG. 15 is a perspective view of a third example of a cardiovascular implant device.

FIG. 16 is a sectional view of a heart illustrating an example positioning at a valve site of the third example of the cardiovascular implant device.

FIG. 17 is a sectional view of a heart illustrating an example positioning at a non-valve site of the third example of the cardiovascular implant.

FIG. 18 is a sectional view of a heart illustrating an example positioning of a fourth example of a cardiovascular implant device.

FIG. 19 is a sectional view of a heart illustrating an example positioning of a fifth example of a cardiovascular implant device.

FIG. 20A is an exploded side view of an expandable sheath device assembly.

FIG. 20B is a side view of an expandable sheath device assembly with a braided layer.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used in this application and in the claims, the singular forms “a,” “an,” and “the” include the plural forms unless the context clearly dictates otherwise. Thus, for example, reference to a “yarn” includes aspects having two or more such yarns unless the context clearly indicates otherwise.

Still further, as used herein, the term “at least one” encompasses one or more of the specified elements. That is, if two of a particular element are present, one of these elements is also present, and thus, “an” element is present.

It is also to be understood that the terminology used herein is for the purpose of describing particular aspects only and is not intended to be limiting. As used in the specification and in the claims, the term “comprising” can include the aspects “consisting of” and “consisting essentially of.” Additionally, the term “includes” means “comprises.”

For the terms “for example,” “exemplary,” and “such as,” and grammatical equivalences thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

All disclosed values also include values that fall within ±10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less' and should be interpreted to include the specific ranges of ‘x,’ ‘y,’ ‘z,’ ‘about x,’ ‘about y,’ and ‘about z’ as well as the ranges of ‘less than x,’ ‘less than y,’ or ‘less than z,’ or ‘less than about x,’ ‘less than about y,’ and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x,’ ‘greater than y,’ ‘greater than z,’ or ‘greater than about x,’ greater than about y,’ ‘greater than about z.’ In addition, the phrase “‘x’ to ‘y’,” where ‘x’ and ‘y’ are numerical values, also includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range. In still further aspects, when the specific values are disclosed between two end values, it is understood that these end values can also be included.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

As used herein, the terms “optional” or “optionally” mean that the subsequently described event or circumstance may or may not occur and that the description includes instances where said event or circumstance occurs and instances where it does not.

Further, the terms “coupled” and “associated” generally mean electrically, electromagnetically, and/or physically (e.g., mechanically, or chemically) coupled or linked and do not exclude the presence of intermediate elements between the coupled or associated items.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,” “on” versus “directly on”). As used herein, the term “and/or” includes any and all combinations of one or more of the associated listed items.

In still further aspects, when the range is given, and exemplary values are provided, it is understood that any ranges can be formed between any exemplary values within the broadest range.

It will be understood that although the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or a section. Thus, a first element, component, region, layer, or section discussed below could be termed a second element, component, region, layer, or section without departing from the teachings of exemplary aspects.

Spatially relative terms, such as “beneath,” “below,” “lower,” “above,” “upper,” “inner,” “outer,” and the like, may be used herein for ease of description to describe one element or feature's relationship to another element(s) or feature(s) as illustrated in the figures, but are not intended to be limiting. It will be understood that the spatially relative terms are intended to encompass different orientations of the device in use or operation, in addition to the orientation depicted in the figures. Even further, certain terms such as “outside,” “inside,” “interior,” and “exterior” can also provide some clarity with relative relationships, but such terms are not intended to imply absolute relationships, positions, and/or orientation. For example, if the device in the figures is turned over, elements described as “below,” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

As used herein, “and/or” means “and” or “or” as well as “and” and “or.”

Still further, as used herein, with reference to implantable device and delivery apparatus, the term “proximal” refers to a position, direction, or portion of a component that is closer to the user and/or a handle of the delivery apparatus that is outside the patient, while “distal” refers to a position, direction, or portion of a component that is further away from the user/or the handle of the delivery apparatus and close to the implantation site. The terms “longitudinal” and “axial” refer to an axis extending from the proximal to and distal directions unless otherwise expressly defined. Further, the term “radial” refers to a direction that is arranged perpendicular to the axis and points along a radium from a center of an object (where the axis is positioned at the center, such as the longitudinal axis of the prosthetic valve).

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to.” However, it should be understood that an appropriate effective amount will be readily determined by one of ordinary skill in the art using only routine experimentation.

As used herein, the term “substantially” means that the subsequently described event or circumstance completely occurs or that the subsequently described event or circumstance generally, typically, or approximately occurs.

Still further, the term “substantially” can, in some aspects, refer to at least about at least about 90%, at least about 91%, at least about 92%, at least about 93%, at least about 94%, at least about 95%, at least about 96%, at least about 97%, at least about 98%, at least about 99%, or about 100% of the stated property, component, composition, or other condition for which substantially is used to characterize or otherwise quantify an amount.

In other aspects, as used herein, the term “substantially free,” when used in the context of the presence of a disclosed component, is to indicate that the recited component is not intentionally present.

As used herein, the term “substantially,” in, for example, the context “substantially identical” or “substantially similar” refers to a method or a system, or a component that is at least about 90%, at least about 91%, at least about 92%, at least about 93%, at least about 94%, at least about 95%, at least about 96%, at least about 97%, at least about 98%, at least about 99%, or about 100% by similar to the method, system, or the component it is compared to.

Moreover, for the sake of simplicity, the attached figures may not show the various ways (readily discernable, based on this disclosure, by one of ordinary skill in the art) in which the disclosed system, method, and apparatus can be used in combination with other systems, methods, and apparatuses. Additionally, the description sometimes uses terms such as “produce” and “provide” to describe the disclosed method. These terms are high-level abstractions of the actual operations that can be performed. The actual operations that correspond to these terms can vary depending on the particular implementation and are, based on this disclosure, readily discernible by one of ordinary skill in the art.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein, and to the Figures and their previous and following description.

In general, the disclosure relates to devices comprising at least one memory alloy member and methods of making the same.

In certain aspects, disclosed is a device comprising a memory alloy member formed at least partly from a superelastic shape-memory alloy. In such aspects, the superelastic shape-memory alloy can display reversible stress-induced martensite at a service temperature such that it has a stress-induced martensitic state and an austenitic state. In such exemplary and unlimiting aspects, the shape memory alloy member can have (i) a deformed shape when the alloy is in its stress-induced martensitic state and (ii) a different unstressed shape when the alloy is in its austenitic state.

In still certain aspects, the superelastic shape-memory alloy is a quaternary shape memory alloy. In such aspects, a conventional shape-memory alloy, such as NiTi can be formed by substituting Ni or Ti with different elements depending on the desired performance. Without wishing to be bound by any theory, it is assumed that substitution of Ti or Ni in a NiTi shape memory alloy with additional metallic elements can significantly shift the martensite start and finish temperatures (Mₛ, M_{f}) and the austenite start and finish temperatures (Aₛ, A_{f}), because the transformation behavior is highly sensitive to composition. To obtain alloys that remain functional at a desired transformation temperature and applied stress, in certain exemplary and unlimiting aspects, the composition needs to be carefully designed by introducing elements that counterbalance these shifts and tailor the Mₛ, M_{f}, Aₛ, and A_{f} values to a targeted range suitable for the intended application. Yet in other aspects, the desired properties can be at least partially controlled with appropriate thermomechanical processing.

In certain aspects, disclosed herein is the superelastic shape-memory alloy comprising, in atomic percent: a) nickel, b) titanium, c) a first element, wherein the first element is present in an amount effective to impart radiopaque properties to the memory alloy element; and d) a second element wherein the second element is present in an amount effective to balance an optional change in a martensite start temperature Mₛ and an austenite finish temperature A_{f} that occurs due to the presence of the first element and to reach a predetermined transformation temperature.

It is understood that in certain aspects, the optional change can be an increase in a martensite start temperature Mₛ and an austenite finish temperature A_{f} that occurs due to the presence of the first element and to reach a predetermined transformation temperature.

Yet in other aspects, the second element can be added to balance other properties of the shape memory alloy, not only the changes in the martensite start temperature Mₛ and/or an austenite finish temperature A_{f}.

In certain aspects, the first element can be Hf. Without being bound by theory, it is understood that, due to its high atomic number, Hf exhibits strong X-ray attenuation and therefore can provide the desired radiopacity. This property can be advantageous when the devices disclosed herein are to be visualized under X-ray or CT imaging.

In certain aspects, Hf can be present in any effective amount that imparts radiopaque properties to the shape memory alloy member. While additions of Hf can be beneficial from radiopaque properties, it is understood that addition of Hf can shift the martensite start and finish temperatures (Mₛ, M_{f}) and the austenite start and finish temperatures (Aₛ, A_{f}) over a broad range, enabling alloys that operate from well below room temperature up to several hundred degrees Celsius. Depending on the application, this change in transformation temperatures can be undesirable. To balance the change the alloys disclosed herein can comprise a second element. In such exemplary and unlimiting aspects, the second element can comprise Al. In such exemplary and unlimiting aspects, Al can be introduced in an effective amount to moderate the transformation-temperature changes caused by Hf. By partially substituting Al on the Ni/Ti lattice sites, the overall electron concentration and lattice parameters can be adjusted so that the increases in Mₛ and A_{f} induced by Hf are balanced, yielding alloys with transformation temperatures and hysteresis better matched to the targeted application.

In certain aspects, the superelastic shape-memory alloy disclosed herein can comprise: a) 49-51 at % of Ni, b) 28-50 at % of Ti; wherein c) the first element is Hf present in an amount of 0.1-20 at %; and d) the second element is Al present in an amount of 0.1-3.5 at %.

For example, the alloy can comprise Ni in an amount of 49-51 at %, including exemplary values of 49.1, 49.2, 49.3, 49.4, 49.5, 49.6, 49.7, 49.8, 49.9, 50.0, 50.1, 50.2, 50.3, 50.4, 50.5, 50.6, 50.7, 50.8, and 50.9 at %. In still further aspects, Ni can be present in any amount that falls within any two foregoing values or within a range formed by any two foregoing values. For example, and without limitations, Ni can be present in an amount of 49-51 at %, 49.2-51 at %, 49.5-51 at %, 49.8-51 at %, 50-51 at %, 50.2-51 at %, 50.5-51 at %, 50.8-51 at %, 49-50.8 at %, 49-50.5 at %, 49-50.2 at %, 49-49.8 at %, 49-49.5 at %, and so on.

In further aspects, the alloy can comprise Ti in an amount of 28-50 at %, including exemplary values of 28.5, 29, 29.5, 30, 30.5, 31, 31.5, 32, 32.5, 33, 33.5, 34, 34.5, 35, 35.5, 36, 36.5, 37, 37.5, 38, 38.5, 39, 39.5, 40, 40.5, 41, 41.5, 42, 42.5, 43, 43.5, 44, 44.5, 45, 45.5, 46, 46.5, 47, 47.5, 48, 48.5, 48, and 49.5 at %. In still further aspects, Ti can be present in any amount that falls within any two foregoing values or within a range formed by any two foregoing values. For example, and without limitations, Ti can be present in an amount of 28-50 at %, 28-49 at %, 28-48 at %, 28-47 at %, 28-46 at %, 28-45 at %, 28-44 at %, 28-43 at %, 28-42 at %, 28-41 at %, 28-40 at %, 28-39 at %, 28-38 at %, 28-37 at %, 28-35 at %, 28-33 at %, 28-30 at %, 28-29 at %, 29-50 at %, 30-50 at %, 33-50 at %, 35-50 at %, 38-50 at %, 40-50 at %, 42-50 at %, 45-50 at %, 48-50 at %, and so on.

In further aspects, the alloy can comprise Hf in an amount of 0.1-20 at %, including exemplary values of 0.1, 0.2, 0.5, 0.7, 1, 1.2, 1.5, 1.8, 2, 2.5, 3, 3.5, 4, 4.5, 5, 5.5, 6, 6.5, 7, 7.5, 8, 8.5, 9, 9.5, 10, 10.5, 11, 11.5, 12, 12.5, 13, 13.5, 14, 14.5, 15, 15.5, 16, 16.5, 17, 17.5, 18, 18.5, 19, and 19.5 at %. In still further aspects, Hf can be present in any amount that falls within any two foregoing values or within a range formed by any two foregoing values. For example, and without limitations, Hf can be present in an amount of 0.1-20 at %, 0.1-18 at %, 0.1-15 at %, 0.1-12 at %, 0.1-10 at %, 0.1-8 at %, 0.1-5 at %, 0.1-2 at %, 0.1-1 at %, 0.1-0.5 at %, 0.5-20 at %, 1-20 at %, 5-20 at %, 10-20 at %, and so on.

In further aspects, the alloy can comprise Al in an amount of 0.1-3.5 at %, including exemplary values of 0.1, 0.2, 0.5, 0.7, 1, 1.2, 1.5, 1.8, 2, 2.5, 3, and 3.2 at %. In still further aspects, Al can be present in any amount that falls within any two foregoing values or within a range formed by any two foregoing values. For example, and without limitations, Hf can be present in an amount of 0.1-3.5 at %, 0.1-3.2 at %, 0.1-3 at %, 0.1-2.8 at %, 0.1-2.5 at %, 0.1-2.2 at %, 0.1-2 at %, 0.1-1.8 at %, 0.1-1.5 at %, 0.1-1.2 at %, 0.1-1 at %, 0.1-0.8 at %, 0.1-0.5 at %, 0.2-3.5 at %, 0.5-3.5 at %, 0.8-3.5 at %, 1-3.5 at %, 1.2-3.5 at %, 1.5-3.5 at %, 1.8-3.5 at %, 2-3.5 at %, 2.5-3.5 at %, 3-3.5 at %, and so on.

In still further aspects, the disclosed herein alloy can comprise Ni₄₉₋₅₁Ti ₂₈₋₃₈Hf₁₀₋₁₉Al _{0.1-3.5}. Yet in other aspects, the alloy can comprise Ni₅₀Ti₃₀₋₃₈Hf₁₁₋₁₉Al₁₋₃. In yet other aspects, the alloy can comprise Ni₅₀Ti₃₆Hf₁₂Al₂. In still further aspects, the alloy can comprise Ni₅₀Ti₃₃Hf₁₅Al₂.

Without wishing to be bound by any theory, it is understood that in shape memory alloys, recoverable stress refers to the internal stress that can be generated and released reversibly as the material transforms between martensite and austenite, enabling the alloy to return to its original shape under load. When the applied stress reaches the critical level for stress-induced martensitic transformation, the stress-strain curve enters a nearly flat stress plateau, during which large strains accumulate at approximately constant stress as austenite converts to martensite and, upon unloading or heating, transforms back to austenite to produce recoverable deformation.

In certain aspects, to achieve a desirable stress-induced behavior, the shape-memory alloy used herein is thermomechanically processed such that: (i) it exhibits superelastic properties at least at the service temperature, (ii) the alloy is substantially austenitic at zero applied stress at the service temperature, (iii) application of mechanical stress at the service temperature induces a martensitic phase transformation over a strain range, with austenite to martensite transformation stresses in a range of 500-2500 MPa (e.g., 550-2500 MPa, 600-2500 MPa, 650-2500 MPa, 700-2500 MPa, 750-2500 MPa, 800-2500 MPa, 850-2500 MPa, 900-2500 MPa, 950-2500 MPa, 1000-2500 MPa, 1050-2500 MPa, 1100-2500 MPa, 1150-2500 MPa, 1200-2500 MPa, 1250-2500 MPa, 1300-2500 MPa, 1350-2500 MPa, 1400-2500 MPa, 1450-2500 MPa, 1500-2500 MPa, 1550-2500 MPa, 1600-2500 MPa, 1650-2500 MPa, 1700-2500 MPa, 1750-2500 MPa, 1800-2500 MPa, 1850-2500 MPa, 1900-2500 MPa, 1950-2500 MPa, 2000-2500 MPa, 2050-2500 MPa, 2100-2500 MPa, 2150-2500 MPa, 2200-2500 MPa, 2250-2500 MPa, 2300-2500 MPa, 2350-2500 MPa, 2400-2500 MPa, 2450-2500 MPa, 500-2400 MPa, 500-2300 MPa, 500-2200 MPa, 500-2100 MPa, 500-2000 MPa, 500-1900 MPa, 500-1800 MPa, 500-1700 MPa, 500-1600 MPa, 500-1500 MPa, 500-1400 MPa, 500-1300 MPa, 500-1200 MPa, 500-1100 MPa, 500-1000 MPa, 500-900 MPa, 500-800 MPa, 500-700 MPa, and so on); (iv) removal of the applied stress at the service temperature causes the stress-induced martensite to reversibly transform back to austenite without application of external heating; and wherein the shape memory alloy member exhibits a recoverable strain of 4 to 10% (e.g., 4, 5, 6, 7, 8, 9, and 10%, or 4 to 10%, 5 to 10%, 6 to 10%, 7 to 10%, 8 to 10%, 9 to 10%, 4 to 9%, 4 to 8%, 4 to 6%, and so one) when tested at the service temperature.

In yet other aspects, the recoverable strain of the shape memory alloy member, is at least 4%, at least 5%, at least 6%, at least 7%, and so on.

In still further aspects, the shape memory alloy member exhibits a minimum elongation of 4, 5, 6, 8, 10, 11, 12, 13, 14, 15, 16, 17, or 18%, and so on, when tested at the service temperature.

In still further aspects, the device can be any device that needs to have reversible stress-induced behavior and to be visualized with X-ray and CT-scans. Yet in certain aspects, the device is an implantable medical device. In aspects, when the device is medical device, the service temperature is at a body temperature. Yet in other aspects, the device disclosed herein operates at the service temperature in a range 0 to 40° C., including exemplary values of 1, 2, 5, 10, 12, 15, 18, 20, 21, 22, 23, 24, 25, 26, 27, 28, 29, 30, 31, 32, 33, 34, 35, 36, 37, 38, and 39° C. In still further aspects, the service temperature can have any value that falls within any two foregoing values or within a range formed by any two foregoing values. For example, and without limitations, the service temperature can temperature in a range of 0 to 40° C., 5 to 40° C., 10 to 40° C., 15 to 40° C., 20 to 40° C., 22 to 40° C., 25 to 40° C., 30 to 40° C., 32 to 40° C., 33 to 40° C., 34 to 40° C., 35 to 40° C., 36 to 40° C., 37 to 40° C., 38 to 40° C., 0 to 39° C., 0 to 38° C., 0 to 37° C., 0 to 36° C., 0 to 35° C., 0 to 34° C., 0 to 33° C., 0 to 32° C., 0 to 31° C., 0 to 30° C., 0 to 29° C., 0 to 28° C., 0 to 27° C., 0 to 26° C., 0 to 25° C., 0 to 24° C., 0 to 23° C., 0 to 22° C., 0 to 21° C., 0 to 20° C., 0 to 19° C., 0 to 18° C., 0 to 17° C., 0 to 16° C., 0 to 15° C., 0 to 10° C., 0 to 5° C., or 0 to 2° C. Yet in other aspects, the service temperature can temperature in a range of 32 to 40° C., 33 to 40° C., 34 to 40° C., 35 to 40° C., 36 to 40° C., 37 to 40° C., 38 to 40° C., 32 to 39° C., 32 to 38° C., 32 to 37° C., 32 to 36° C., 32 to 35° C., and so on.

In still further aspects, the device can comprise a shunt, a stent, a valve device, a prosthetic valve device, a docking station, a pre-stent, an edge-to-edge valve repair device, a valve delivery system, a peripheral arterial or coronary stent, a neurovascular stent, a left atrial appendage closure, inferior vena cava filter, a catheter, a surgical instrument, an orthopedic implant, an endovascular aneurysm graft, neurovascular coil, bone anchor or screw, retriever or grasper, intrauterine device (IUD) bone fixation plates, staples, nails, spinal correction rods, bone clips and compression devices, orthodontic archwires, braces, endodontic devices, gastrointestinal tools (e.g., endoscopes, kink-resistant surgical instruments and the like), and combinations thereof, and wherein the device is configured to induce and remove the stress.

Yet still in further aspects, the shape memory alloy member can comprise a valve frame, a reinforcing member of a sheath, a guide wire, a catheter pull wire, a catheter body, a catheter shaft, or any combination thereof. In certain exemplary and unlimiting aspects, the reinforcing layer is a braided layer.

Some exemplary devices and shape memory alloy members are described in detail below and shown in the enclosed Figures.

It is understood that the shape memory alloy member can have any desirable thickness. It is understood that in some aspects, it is desirable to have a small profile as possible, and therefore it is desirable to have members with a low thickness that still provide the desired radiopaque effect.

In certain aspects, the shape memory alloy member has a thickness in a range of 50 to 250 micrometers, 50 to 225 micrometers, 50 to 200 micrometers, 50 to 150 micrometers, 50 to 100 micrometers, 75 to 250 micrometers, 100 to 250 micrometers, 150 to 250 micrometers, and so on.

In still further aspects, the shape memory alloy member exhibits 20-200% (e.g., 20-200%, 20-180%, 20-150%, 20-120%, 20-100%, 20-80%, 20-50%, 50-200%, 80-200%, 100-200%, 120-200%, 150-200%, and so on increase in radiopacity as compared to a substantially identical reference memory alloy member formed from NiTi alloy. It is understood that the changes in radiopacity are measured as described below.

In yet other aspects, in addition or in the alternative, the shape memory alloy member exhibits a 30-400% (e.g., 30-400%, 30-350%, 30-300%, 30-250%, 30-200%, 30-150%, 30-100%, 30-50%, 50-400%, 100-400%, 150-400%, 200-400%, and so on) increase in strength as compared to a substantially identical reference memory alloy member formed from NiTi alloy.

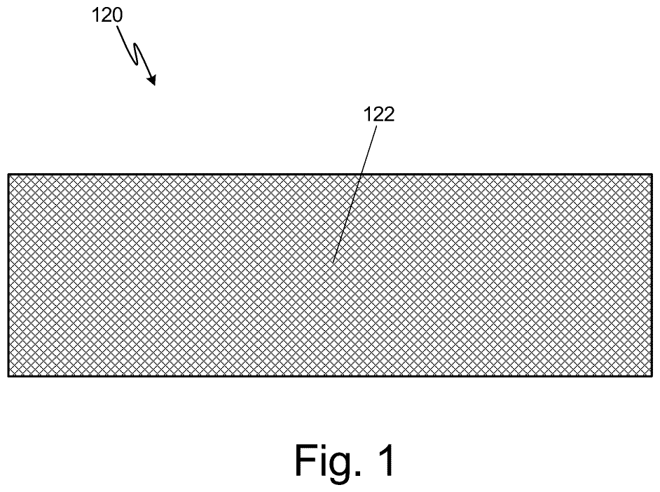

FIGS. 1-7 illustrate the disclosed herein devices and steps of making the same.

### Implantable Medical Device 120

FIG. 1 is a schematic cross section of implantable medical device 120. Implantable medical device 120 includes alloy 122.

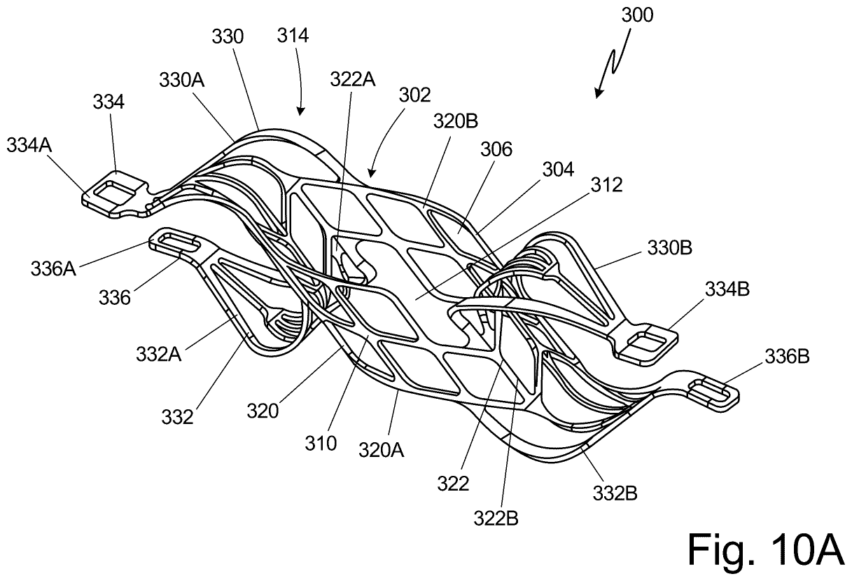

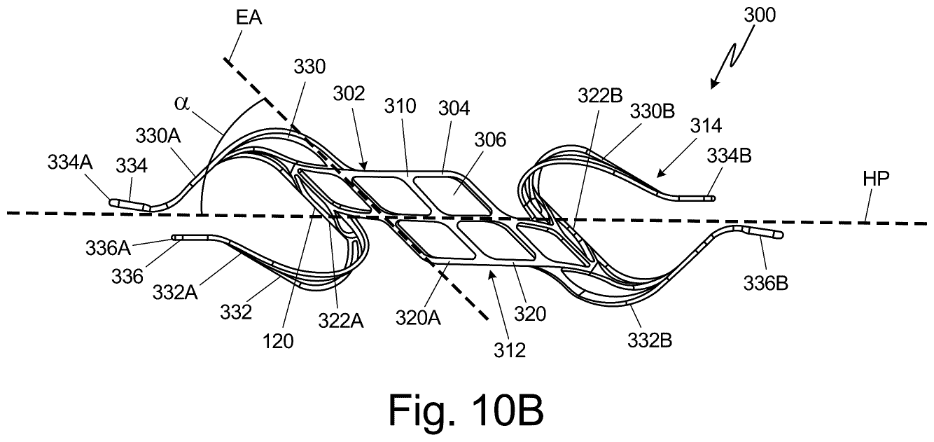

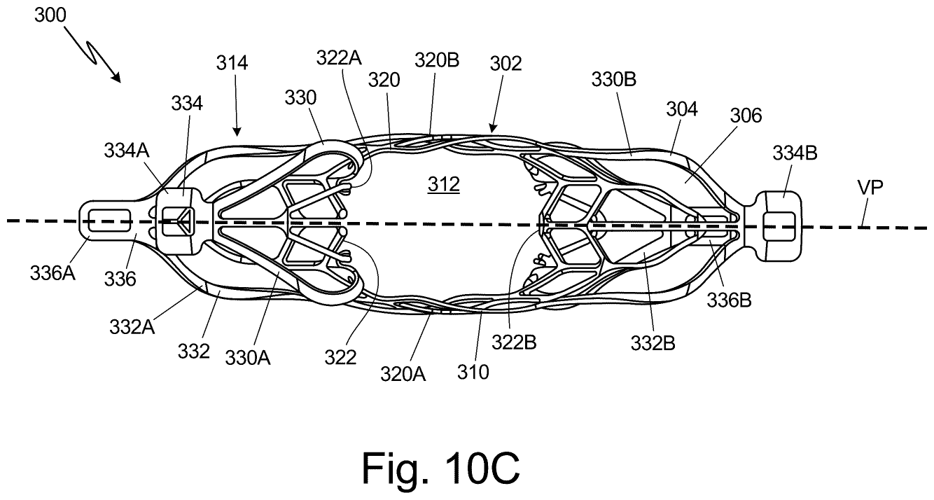

Implantable medical device 120 is a schematic representation of a cross-section of an implantable medical device made from alloy 122. Implantable medical device 120 can be any implantable medical device of any shape or size, for example, shunts, heart valve frames, heart therapy devices, and markers. Further, the cross-section shown in FIG. 1 can be a representation of a section or portion of implantable medical device 120. In the following discussion, implantable medical device 120 will be used as an example of a device that can be made from alloy 122. However, implantable medical device 120 can be any suitable medical device, including any of implantable medical devices 300, 400, 500, 600, 700, and 800 shown in FIGS. 10A-19. Implantable medical device 120 can have any suitable size. In one aspect, implantable medical device 120 is between 50 micrometers and 250 micrometers thick. In another aspect, implantable medical device 120 is between 100 micrometers and 180 micrometers thick. In yet another aspects, implantable medical device 120 can have any of disclosed above thicknesses or can fall within any of the disclosed above ranges.

Implantable medical device 120 is made from an alloy 122, which has radiopaque and superelastic qualities. Alloy 122 is a quaternary alloy comprising NiTiHfAl, which is superelastic and has shape-memory properties, having any of the disclosed above compositions. Nickel-titanium alloys (often referred to as Nitinol) are known alloys possessing superelastic and shape-memory properties. Nitinol alloys also have high strength, are corrosion resistant, and are biocompatible, making them good choices for the manufacture of medical devices. However, Nitinol alloys may not have sufficient radiopacity, and it can be challenging to visualize medical devices, and in particular small medical devices, made from Nitinol alloys under X-ray fluoroscopy or other visualization techniques used during clinical procedures. This is particularly true for small implantable medical devices.

Alloy 122 includes the addition of hafnium and aluminum to improve the radiopacity of alloy 122 while maintaining the superelastic and shape-memory properties desired for implantable medical devices. The hafnium in alloy 122 has radiopaque properties that increases the radiopacity of alloy 122 and thus implantable medical device 120. Hafnium increases radiopacity due to its higher atomic number (Z), allowing for more contrast in X-ray fluoroscopy imaging. However, the hafnium in alloy 122 increases the minimum temperature at which superelasticity is observed to levels beyond human body temperature. The aluminum in alloy 122 allows medical device 120 to have superelasticity at or near human body temperature. Aluminum contributes to higher and tunable mechanical properties through the formation of precipitates (such as the Heusler phase with L21 crystal structure). Using alloy 122 for implantable medical device 120 increases the radiopacity of implantable medical device 120 compared to implantable medical devices made of a binary NiTi alloy (nitinol), while allowing implantable medical device 120 to maintain its shape-memory characteristics at or near human body temperature. Alloy 122 also has tunable mechanical properties through slight compositional changes or differing heat treatments.

In a first unlimited example, alloy 122 can be a NiTiHfAl quaternary alloy including nickel between 49-51 atomic percent (at. %), titanium between 28-38 at. %, hafnium between 10-18 at. %, and aluminum between 0.1-3.5 at. %. In a second example, alloy 122 can be a NiTiHfAl quaternary alloy including nickel at 50 at. %, titanium between 30-38 at. %, hafnium between 11-19 at. %, and aluminum between 1-3 at. %. Specifically, in a first example, an alloy with a composition of Ni₅₀Ti₃₆Hf₁₂Al₂ is disclosed. Further, in a second example, an alloy with a composition of Ni₅₀Ti₂₉Hf₁₈Al₃ is disclosed. In a third example, an alloy with a composition of Ni₅₀Ti₃₃Hf₁₅Al₂ is disclosed. In a fourth example, an alloy with a composition of Ni₅₀Ti_{37.5}Hf₁₅Al_{0.5} is disclosed. In a fifth example, an alloy with a composition of Ni₅₀Ti₃₇Hf₁₅Al₁ is disclosed. Hafnium between 10 and 19 atomic percent avoids excess precipitation strengthening in the alloy and promotes workability of the alloy to allow the alloy to be drawn into a wire, bar/rod, tube, sheet, or strip form. Higher atomic percentages of both hafnium and aluminum in a quaternary alloy would result in precipitation, hampering the wire/tube/sheet/strip/bar/rod drawing process.

An increase in the radiopacity of implantable medical device 120 using alloy 122 helps with the visualization of implantable medical device 120 during clinical procedures, and specifically during placement of the device. This can reduce embolization risk and result in better clinical outcomes. Further, implantable medical device 120 can be made smaller in volume or thickness in some instances due to the increased radiopacity of alloy 122. Additionally, an alloy with increased mechanical properties offers a higher radial resistive force and chronic outward force, lower push forces, and can enable thinner/smaller device thicknesses and smaller crimp profiles. The tunable mechanical properties of alloy 122 allows for tuning of mechanical properties based on the function of implantable medical device 120. For example, implantable medical device 120 can be tuned to have higher fatigue strength.

Using alloy 122 to manufacture implantable medical device 120 can simplify manufacturing by eliminating the need for additional visualization markers and making the manufacturing easier, faster, and cheaper. Further, the strut dimensions of implantable medical device 120 and the crimp profile of implantable medical device 120 can be decreased. Additionally, the mechanical properties of implantable medical device 120 can be tuned while achieving full device visibility.

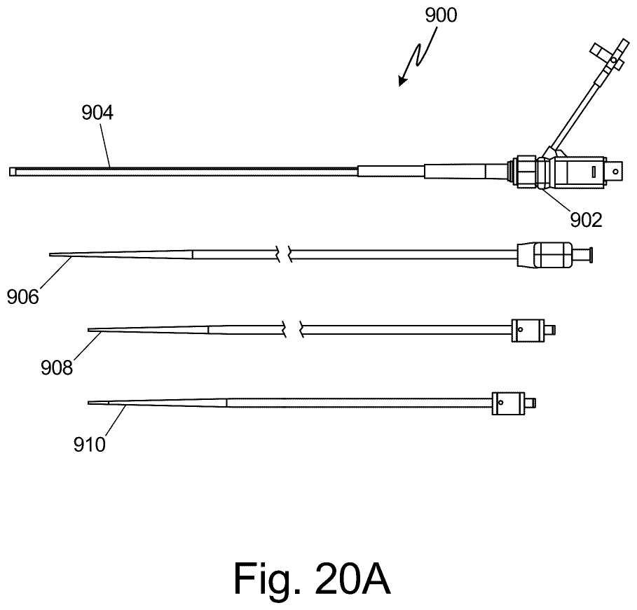

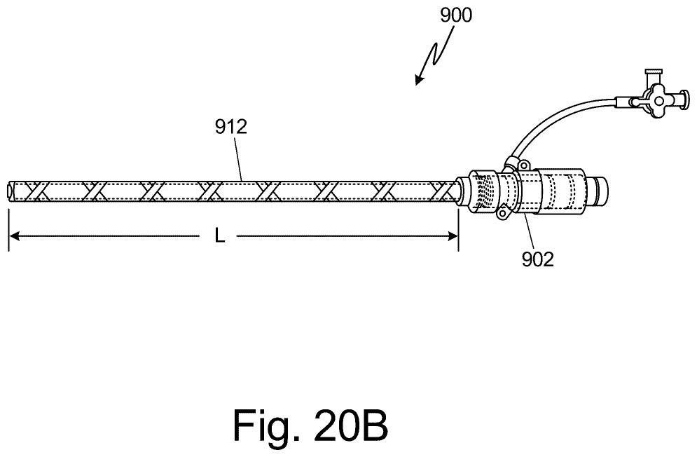

Alloy 122 has been discussed herein in the context of an implantable medical device. However, alloy 122 can also be used in the manufacture and design of delivery system components for medical devices, for example sheaths, guidewires, actuation wires, connectors, nuts, and markers. Examples of delivery system components are shown in FIGS. 20A-20B. The tunable mechanical properties of alloy 122 also allows tuning of the mechanical properties of any delivery system component. For example, if alloy 122 can be tuned to have higher ultimate tensile strength if alloy 122 is used for an actuation wire of a delivery system.

Examples of implantable medical devices include shunts, stents, and docking stations. Additionally, the alloy disclosed herein can also be used in the manufacture and design of delivery system components for medical devices, as described in relation to FIGS. 20A-20B. Example delivery system components include sheaths, guidewires, actuation wires, connectors, nuts, and markers. In one example, the delivery system component can include a pull ring for a delivery system for a shunt device, a sheath for a delivery system for a transcatheter heart valve, and a wire of a moveable arm linkage for a delivery system for a shunt device.

In still further aspects, the device can be an implantable medical device comprising a shunt, a stent, a valve device, a valve delivery device, a prosthetic valve device, a docking station, a pre-stent, an edge-to-edge valve repair device, and combinations thereof, and wherein the device is configured to induce and remove the stress.

Also disclosed herein are methods comprising forming the shape memory alloy member of the device of any one of the examples herein. In certain aspects, the shape memory alloy member is formed from an ingot of a superelastic shape-memory alloy comprises, in atomic percent: a) nickel, b) titanium, c) a first element, wherein the first element is present in an amount effective to impart radiopaque properties to the memory alloy element; and d) a second element, wherein the second element is present an amount effective to balance an optional change in a martensite start temperature Mₛ and finish A_{f} that occurs due to the presence of Hf and to reach a predetermined transformation temperature.

It is understood and as disclosed above, the first element can be Hf and the second element can be Al. It is further understood that the superelastic shape-memory alloy can comprise any of the disclosed compositions and ratios of the Ni, Ti, Hf, and Al.

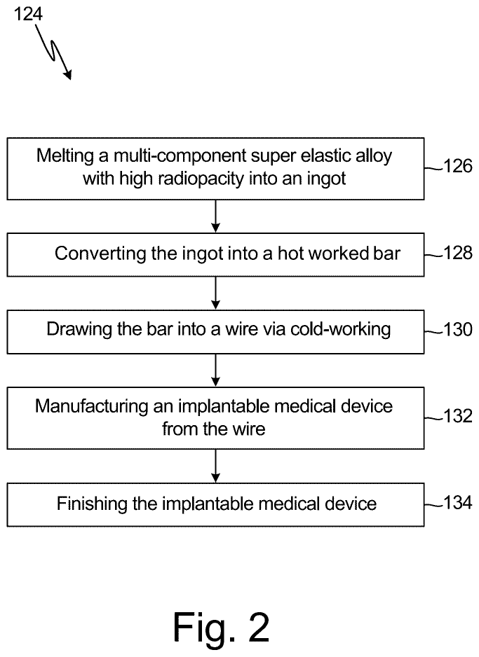

FIG. 2 is a flow chart showing method 124 of using wrought methods to create a wire that can be made into an implantable medical device 120 from alloy 122, as described in FIG. 1. Method 124 includes step 126, step 128, step 130, step 132, and step 134.

In certain aspects, the disclosed herein superelastic alloy is melted into an ingot (Step 126). The ingot can be formed by any known in the art methods. For example, and without limitations, the ingot can be formed by melting the superelastic shape-memory alloy via vacuum induction melting (VIM), vacuum arc melting (VAR), plasma arc melting (PAM), or a combination thereof.

In yet still further aspects, the melted superelastic shape memory alloy is homogenized at a temperature of 800-1500° C. (e.g. 800-1500° C., 900-1500° C., 1000-1500° C., 1100-1500° C., 1200-1500° C., 1300-1500° C., 1400-1500° C., 800-1400° C., 800-1300° C., 800-1200° C., 800-1100° C., 800-1000° C., 1000-1400° C., and so on) for 12 to 96 hours (e.g., 12 to 84 hours, 12 to 72 hours, 12 to 60 hours, 12 to 48 hours, 12 to 36 hours, 24 to 96 hours, 36 to 96 hours, 48 to 96 hours, 60 to 96 hours, 72 to 96 hours, 36 to 72 hours, 48 to 72 hours, and so on) prior to forming the ingot.

In still further aspects, the ingot can be formed into a wire, a tube, a sheet, a rod, or any combination thereof. It is understood that before forming the ingot in any of the shapes disclosed above, the ingot can be first formed in any semi-finished products, such as a forged bar or slab, and so on. In still further aspects, the wire, the tube, the sheet, the rod or any combination thereof is thermomechanically processed at 200-700° C. (e.g., 250-700° C., 300-700° C., 350-700° C., 400-700° C., 450-700° C., 500-700° C., 550-700° C., 600-700° C., 650-700° C., 200-650° C., 200-600° C., 200-550° C., 200-500° C., 200-450° C., 200-400° C., 200-300° C., 450-600° C., and so on) prior to forming the memory alloy element.

An exemplary methods steps are further shown in FIG. 2. For example, step 128 includes converting the ingot into a hot-worked bar. Step 128 can be accomplished through any reasonable method. For example, the ingot may be converted into a hot-worked bar via hot extrusion or hot forging.

Step 130 includes drawing the hot-worked bar into a wire via cold-working. Alloys with higher hafnium percentages than the alloys disclosed in FIG. 1 may have precipitation hardening, which may cause the wire to break before it is sufficiently reduced in diameter via cold-working. Alloy 122 with lower hafnium percentages can be drawn into a wire via cold-working without causing the wire to break. The wire can be heat treated at a temperature of 450-600° C. to impart superelasticity. It is further understood that the temperature range can be changed to arrive at the desired properties.

Step 132 includes manufacturing an implantable medical device from the wire. Step 132 can include forming the implantable medical device with the wire. Step 132 can also include shape-setting or heat treating the implantable medical device form. Shape-setting and heat treating are processes to “train” shape memory and super-elastic materials into the form necessary for the target implantable medical device. Shape-setting and heat treating can include heating and/or cooling the implantable medical device form. The wire can be heat treated in the range of 200-700° Celsius to impart superelasticity with an upper plateau stress in the range of 800-2500 MPa, a recoverable strain of at least 6%, and a minimum elongation of 10%, when measured using tensile test bars at 37° Celsius. It is understood that the stress at strain can be measured per ASTM F2516. In certain aspects, the fracture elongation can be about 7% for the composition comprising 18% at Hf when it was subjected to 480° C. treatment.

It is understood that the shape memory alloy member can have any of the thicknesses disclosed above.

In certain aspects, the shape memory alloy member is surface-treated prior to incorporating it into the device, wherein the surface treatment comprises surface finishing and/or sterilization. Yet in still further aspects, the surface finishing process is chosen from the group consisting of mechanical polishing, chemical etching, chemical polishing, electropolishing, and combinations thereof.

For example, as shown in FIG. 2, step 134 includes finishing the implantable medical device. Step 134 results in a finished target implantable device. Step 134 can also include finishing processes including surface finishing and sterilization. Surface finishing processes include mechanical polishing, chemical etching, chemical polishing, electropolishing, and combinations thereof. In a first example, finishing can include electropolishing the shape set wire using an electrolyte to result in a smooth corrosion-resistant surface. Sterilization can be performed through any reasonable sterilization technique.

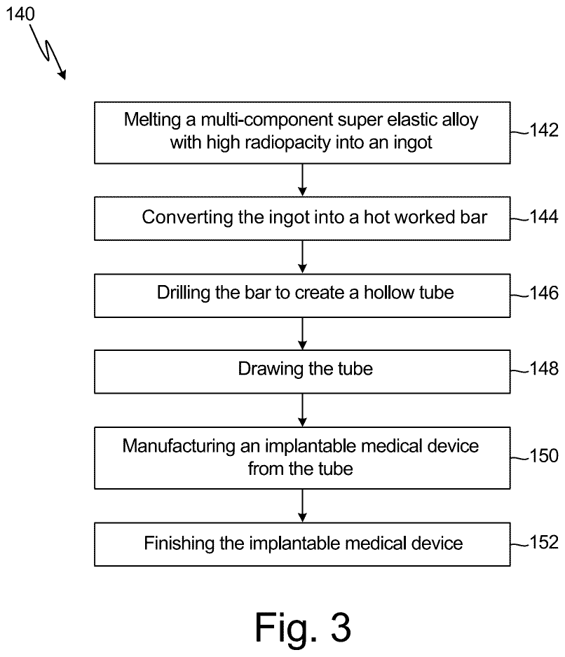

FIG. 3 is another exemplary flow chart showing method 140 of using wrought methods to create a tube that can be used to make an implantable medical device 120 from alloy 122, as described in FIG. 1. Method 140 includes step 142, step 144, step 146, step 148, step 150, and step 152.

An exemplary step 142 includes melting a multi-component superelastic alloy with high radiopacity into an ingot. The superelastic alloy with high radiopacity can be alloy 122 as described with respect to FIG. 1. Alloy 122 can be cast into an ingot form using a melting process under vacuum, such as vacuum induction melting (VIM), vacuum arc remelting (VAR), plasma arc melting (PAM), or a combination of melting processes, and homogenized in the range of 1000-1400 C for 48-72 hours (or at any other disclosed above conditions).

Step 144 includes converting the ingot into a rod. Step 144 can be accomplished through any reasonable methods as disclosed above.

In other exemplary aspects, step 146 can include converting the rod to create a hollow tube. Step 146 can be accomplished through any reasonable method. It is contemplated that the same materials and methods described may be used to create a flat strip or sheet of alloy, rather than a tube or wire.

Step 148 includes drawing the tube. Step 148 increases the length and decreases the width of the tube. The tube produced by step 148 should be the approximate diameter of a target implantable medical device (for example, implantable medical device 120).

Step 150 includes manufacturing an implantable medical device from the tube. Step 150 can include laser cutting the implantable medical device to include any structures (for example, struts and openings) necessary for the target implantable medical device. Step 150 can also include shape-setting or heat treating the implantable medical device. Shape-setting and heat treating are processes to “train” shape memory and super-elastic materials into the necessary form for the target implantable medical device. Shape-setting and heat treating can include heating and/or cooling the implantable medical device.

For example, the laser cut tube can be expanded onto a mandrel and heat treated in the range of 450-700° C. to impart superelasticity with an upper plateau stress in the range of 800-1100 MPa, a recoverable strain of at least 6%, and a minimum elongation of 10%, when measured using tensile test bars at 37° C. It is understood that any of the disclosed above conditions can also be used, and the formed device can exhibit any of the disclosed above properties.

Step 152 includes finishing the implantable medical device. Step 152 results in a finished target implantable device. Step 152 can include finishing processes including surface finishing and sterilization. Surface finishing processes include mechanical polishing, chemical etching, chemical polishing, electropolishing, and combinations thereof. Sterilization can be performed through any reasonable sterilization technique.

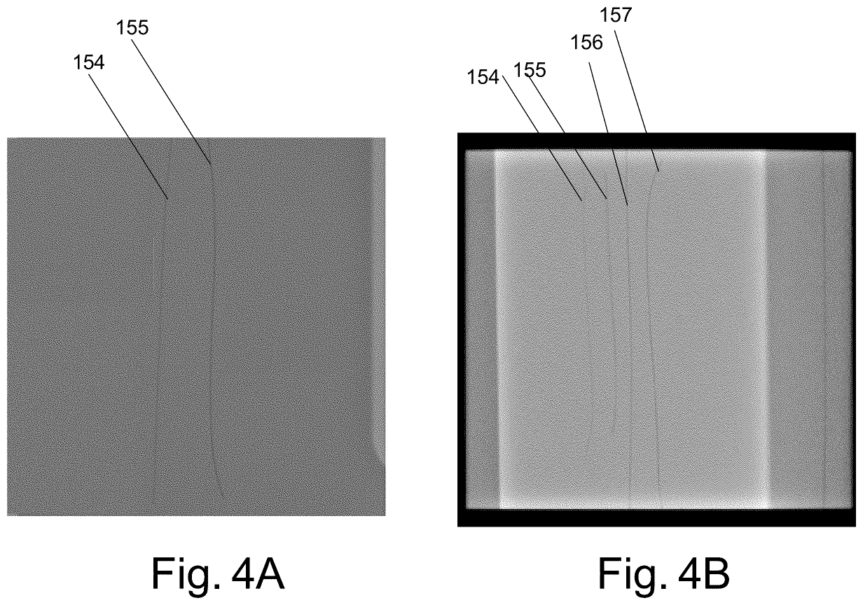

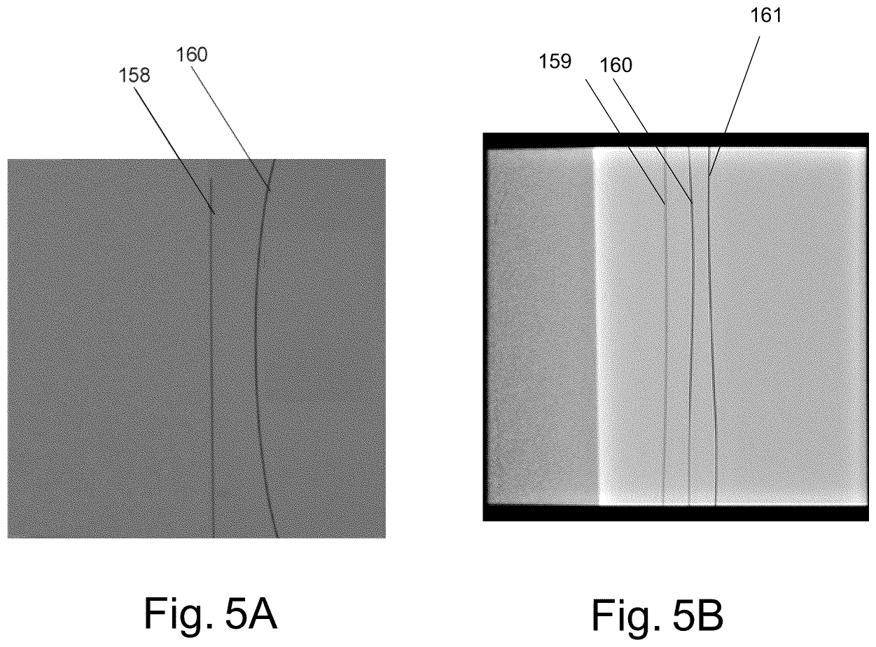

FIGS. 4A-5B shows fluoroscopic images of the devices according to some aspects of the disclosure as compared to traditional memory-shape devices. For example, FIG. 4A shows a first fluoroscopic image comparing the radiopacity of different alloy compositions at a first thickness. While FIG. 4B shows a second fluoroscopic image comparing the radiopacity of different alloy compositions at the first thickness. FIG. 5A is a first fluoroscopic image comparing the radiopacity of different alloy compositions at a second thickness. FIG. 5B is a second fluoroscopic image comparing the radiopacity of different alloy compositions at the second thickness.

FIGS. 4A-5B will be discussed together. FIG. 4A shows an exemplary and unlimiting nickel-titanium alloy wire 154 and Ni₅₀Ti₃₆Hf₁₂Al₂ alloy wire 155. FIG. 4B shows an exemplary and unlimiting nickel-titanium alloy wire 154, Ni₅₀Ti₃₆Hf₁₂Al₂ alloy wire 155, Ni₅₀Ti₃₃Hf₁₅Al₂ alloy wire 156, and Ni₅₀Ti₂₉Hf₁₈Al₃ alloy wire 157. FIG. 5A shows an exemplary and unlimiting nickel-titanium-chromium wire 158 and Ni₅₀Ti₃₆Hf₁₂Al₂ alloy wire 160. FIG. 5B shows an exemplary and unlimiting nickel-titanium alloy wire 159, Ni₅₀Ti₃₆Hf₁₂Al₂ alloy wire 160, and Ni₅₀Ti₃₃Hf₁₅Al₂ alloy wire 161.

FIG. 4A shows a fluoroscopic image of nickel-titanium alloy wire 154 having a diameter of 0.007″ and Ni₅₀Ti₃₆Hf₁₂Al₂ alloy wire 155, also having a diameter of 0.007″. FIG. 4B shows the same wires as depicted in FIG. 4A, and further includes Ni₅₀Ti₃₃Hf₁₅Al₂ alloy wire 156 having a diameter of 0.007″ and Ni₅₀Ti₂₉Hf₁₈Al₃ alloy wire 157 having a diameter of 0.007″. As such, all wires shown in FIGS. 4A and 4B have a diameter of 0.007″. FIG. 5A shows nickel-titanium-chromium wire 158 having a diameter of 0.016″ and Ni₅₀Ti₃₆Hf₁₂Al₂ alloy wire 160 also having a diameter of 0.016″. FIG. 5B shows the Ni₅₀Ti₃₆Hf₁₂Al₂ alloy wire 160 as depicted in FIG. 4B, and further includes nickel-titanium alloy wire 159 having a diameter of 0.016″ and Ni₅₀Ti₃₃Hf₁₅Al₂ alloy wire 161 having a diameter of 0.016″. As such, all wires shown in FIGS. 5A and 5B have a diameter of 0.016″. All of FIGS. 4A-5B show that the wires made out of Ni₅₀Ti₃₆Hf₁₂Al₂, including Ni₅₀Ti₃₆Hf₁₂Al₂ alloy wire 155 and Ni₅₀Ti₃₆Hf₁₂Al₂ alloy wire 160, have improved radiopacity compared to nickel-titanium alloy wire 154 and nickel-titanium-chromium wire 158. The Ni₅₀Ti₃₆Hf₁₂Al₂ alloy used for wires 155 and 160 is able to be drawn into a wire with shape-memory properties that are activated at the temperature of the human body. This alloy has 30-50% more radiopacity compared to nitinol when using ASTM F640 radiopacity determination methods.

When a gray-scale analysis is performed on the wires depicted in FIG. 4A, Ni₅₀Ti₃₆Hf₁₂Al₂ alloy wire 155 has a ratio of difference of 75% when compared to nickel-titanium alloy wire 154. When the same gray-scale analysis is performed on the wire depicted in FIG. 4B, Ni₅₀Ti₃₆Hf₁₂Al₂ alloy wire 155 has a ratio of difference of 73% when compared to nickel-titanium alloy wire 154; Ni₅₀Ti₃₃Hf₁₅Al₂ alloy wire 156 has a ratio of difference of 73% when compared to nickel-titanium alloy wire 154; and Ni₅₀Ti₂₉Hf₁₈Al₃ alloy wire 157 has a ratio of difference of 73% when compared to nickel-titanium alloy wire 154. When the same gray-scale analysis is performed on the wires depicted in FIG. 5A, Ni₅₀Ti₃₆Hf₁₂Al₂ alloy wire 160 has a ratio of difference of 56% compared to nickel-titanium-chromium wire 158. When the same gray-scale analysis is performed on the wires depicted in FIG. 5B, Ni₅₀Ti₃₆Hf₁₂Al₂ alloy wire 160 has a ratio of difference of 53% compared to nickel-titanium alloy wire 159; and Ni₅₀Ti₃₃Hf₁₅Al₂ alloy wire 161 has a ratio of different of 53% compared to nickel-titanium alloy wire 159.

Overall, based on trials, 0.007″ wires of Ni₅₀Ti₃₆Hf₁₂Al₂, Ni₅₀Ti₃₃Hf₁₅Al₂, and Ni₅₀Ti₂₉Hf₁₈Al₃ show a radiopaque improvement of between 50-110% when compared to a binary nitinol 0.007″ wire, and 0.016″ wires of Ni₅₀Ti₃₆Hf₁₂Al₂ and Ni₅₀Ti₃₃Hf₁₅Al₂ show a radiopaque improvement between 45-90% when compared to a binary nitinol 0.016″ wire. All of the Ni₅₀Ti₃₆Hf₁₂Al₂, Ni₅₀Ti₃₃Hf₁₅Al₂, and Ni₅₀Ti₂₉Hf₁₈Al₃ alloys show a measurable and significant radiopacity improvement over binary nickel-titanium.

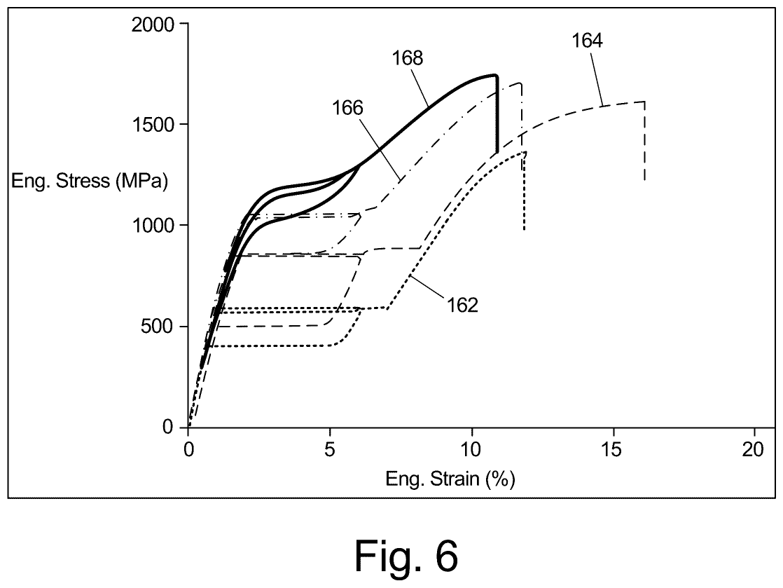

FIG. 6 is a graph that illustrates stress versus strain curves for different alloys. FIG. 6 shows stress strain curve 162, stress strain curve 164, stress strain curve 166, and stress strain curve 168.

FIG. 6 shows stress strain curve 162 for nitinol, stress strain curve 164 for a Ni₅₀Ti₃₆Hf₁₂Al₂ alloy treated at a first heat treatment temperature of 640° C. for 5 minutes, stress strain curve 166 for a Ni₅₀Ti₃₆Hf₁₂Al₂ alloy treated at a second heat treatment temperature of 550° C. for 5 minutes, and stress strain curve 168 for a Ni₅₀Ti₃₆Hf₁₂Al₂ alloy treated at a third heat treatment temperature of 480° C. for 5 minutes. All stress-strain curves 162, 164, 166, 168 were measured at human body temperature. Stress strain curves 164, 166, and 168 exhibit greater plateau strengths, ultimate tensile strengths, and comparable percent uniform elongation and austenitic moduli when compared to stress strain curve 162. Stress strain curves 164, 166, and 168 all show at least a six percent recoverable strain equivalent as compared to nitinol curve 162. Specifically, stress strain curves 164, 166, and 168 shows a six to eight percent recoverable strain. Different heat treatment temperatures of wires corresponding to stress strain curves 164, 166, 168 allow for different amounts of stress depending on the desired characteristics of the implantable medical device or delivery system component.

As shown in FIG. 6, varying the heat treatment of a Ni₅₀Ti₃₆Hf₁₂Al₂ alloy allows for tunable mechanical properties of the Ni₅₀Ti₃₆Hf₁₂Al₂ alloy. This allows the Ni₅₀Ti₃₆Hf₁₂Al₂ alloy to be used in many implantable medical devices and delivery system components, as the mechanical properties of the Ni₅₀Ti₃₆Hf₁₂Al₂ alloy can be tuned based on the properties desired for the implantable medical device or delivery system component.

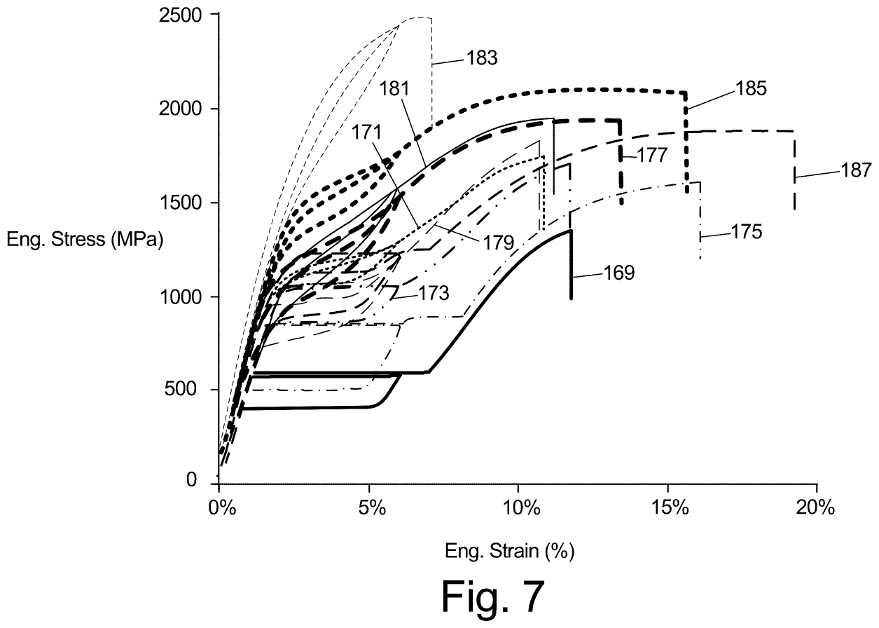

FIG. 7 is a second graph showing engineering stress versus strain curves for different alloys. FIG. 7 shows stress strain curves 169, 171, 173, 175, 177, 179, 181, 183, 185, and 187.

An exemplary and unlimiting aspects are shown in FIG. 7. More specifically, FIG. 7 shows stress strain curve 169 for nitinol, stress strain curve 171 for a Ni₅₀Ti₃₆Hf₁₂Al₂ alloy treated at a first heat treatment temperature of 480° C. for 5 minutes, stress strain curve 173 for a Ni₅₀Ti₃₆Hf₁₂Al₂ alloy treated at a second heat treatment temperature of 550° C. for 5 minutes, stress strain curve 175 for a Ni₅₀Ti₃₆Hf₁₂Al₂ alloy treated at a third heat treatment temperature of 640° C. for 5 minutes, stress strain curve 177 for a Ni₅₀Ti₃₄Hf₁₅Al₂ alloy at a first heat treatment temperature of 480° C. for two minutes, stress strain curve 179 for a Ni₅₀Ti₃₄Hf₁₅Al₂ alloy at a second heat treatment temperature of 550° C. for two minutes, stress strain curve 181 for a Ni₅₀Ti₃₄Hf₁₅Al₂ alloy at a third heat treatment temperature of 640° C. for three minutes, stress strain curve 183 for a Ni₅₀Ti₃₁Hf₁₈Al₂ alloy at a first heat treatment temperature of 480° C. for two minutes, stress strain curve 185 for a Ni₅₀Ti₃₁Hf₁₈Al₂ alloy at a second heat treatment temperature of 550° C. for two minutes, and stress strain curve 187 for a Ni₅₀Ti₃₁Hf₁₈Al₂ alloy at a third heat treatment temperature of 640° C. for two minutes.

As discussed above with regard to FIG. 6, stress strain curves corresponding to metal alloys Ni₅₀Ti₃₆Hf₁₂Al₂ exhibit greater plateau strengths, ultimate tensile strengths, and comparable percent uniform elongation and austenitic moduli when compared to stress strain curves associated with Nitinol. In addition to the improvement over Nitinol shown in FIG. 6 and with alloys composed of Ni₅₀Ti₃₆Hf₁₂Al₂, FIG. 7 shows an improvement over Nitinol and over alloys with lower hafnium percentage with alloys composed of Ni₅₀Ti₃₄Hf₁₅Al₂ and Ni₅₀Ti₃₁Hf₁₈Al₂. Specifically, across all alloys depicted in FIG. 7 as compared to nitinol and stress strain curve 169, stress strain curves 171, 173, 175, 177, 179, 181, 183, 185, and 187 show a minimum increase of lower plateau strength of 35% and a maximum increase of 269%, and a minimum improvement of upper plateau strength of 47% and a maximum improvement of at least 224%. The alloys containing hafnium depicted in FIG. 7 have an increase in ultimate tensile strength of 20% to 83% as compared to nitinol. The alloys containing hafnium depicted in FIG. 7 also an increased uniform elongation of up to 52% as compared to nitinol. It is understood that the increase uniform elongation can be anywhere between 10% to 200% (e.g., 10% to 150%, 10% to 100%, 10% to 50%, 50% to 200%, 100% to 200% and so on). These improvements in strength, along with the tunability provided by using different heat treatment temperature and lengths of time, allow for improved outcomes based on the properties desired for the implantable medical device or delivery system component.

### Heart H and Vascularture V

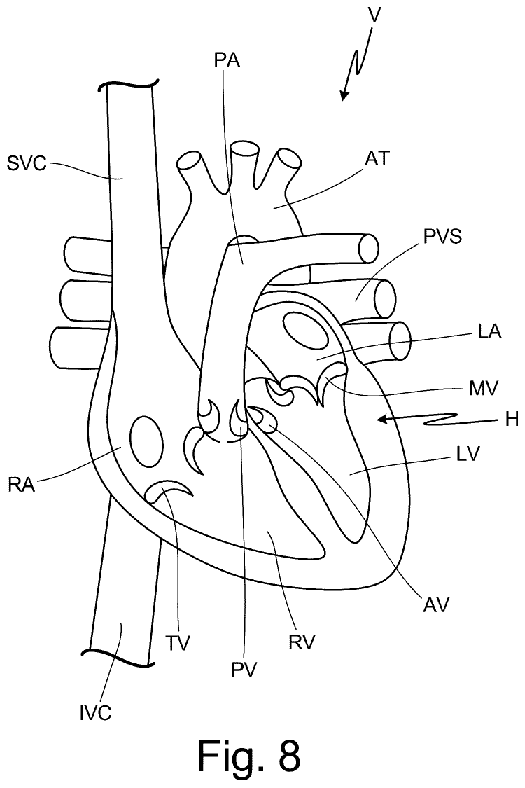

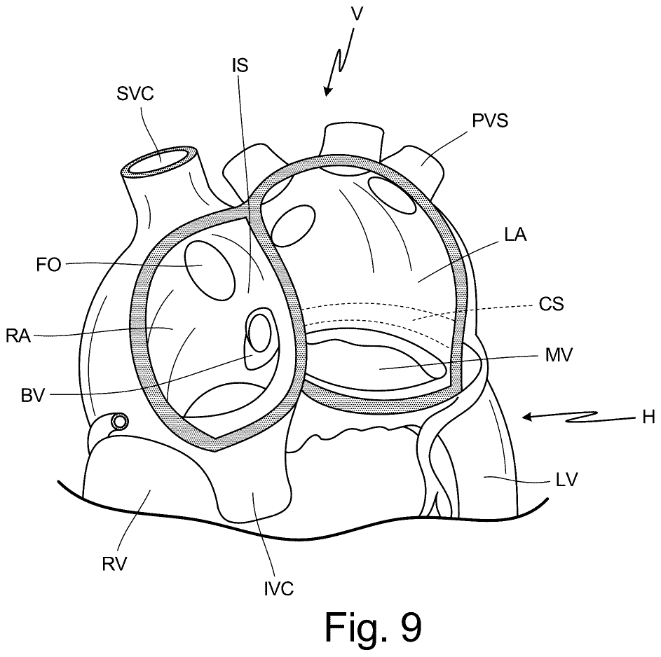

FIG. 8 is a schematic diagram of heart H and vasculature V. FIG. 9 is a cross-sectional view of heart H. FIGS. 8 and 9 will be discussed together. FIGS. 8-9 show heart H, vasculature V, right atrium RA, right ventricle RV, left atrium LA, left ventricle LV, superior vena cava SVC, inferior vena cava IVC, tricuspid valve TV (shown in FIG. 8), pulmonary valve PV (shown in FIG. 8), pulmonary artery PA (shown in FIG. 8), pulmonary veins PVS, mitral valve MV, aortic valve AV (shown in FIG. 8), aorta AT (shown in FIG. 8), coronary sinus CS (shown in FIG. 9), thebesian valve BV (shown in FIG. 9), inter-atrial septum IS (shown in FIG. 9), and fossa ovalis FO (shown in FIG. 9).

Heart H is a human heart that receives blood from and delivers blood to the vasculature V. Heart H includes four chambers: right atrium RA, right ventricle RV, left atrium LA, and left ventricle LV.

The right side of heart H, including right atrium RA and right ventricle RV, receives deoxygenated blood from vasculature V and pumps the blood to the lungs. Blood flows into right atrium RA from superior vena cava SVC and inferior vena cava IVC. Right atrium RA pumps the blood through tricuspid valve TV into right ventricle RV. The blood is then pumped by right ventricle RV through pulmonary valve PV into pulmonary artery PA. The blood flows from pulmonary artery PA into arteries that deliver the deoxygenated blood to the lungs via the pulmonary circulatory system. The lungs can then oxygenate the blood.

The left side of heart H, including left atrium LA and left ventricle LV, receives the oxygenated blood from the lungs and pumps the blood to the body. Blood flows into left atrium LA from pulmonary veins PVS. Left atrium LA pumps the blood through mitral valve MV into left ventricle LV. The blood is then pumped by left ventricle LV through aortic valve AV into aorta AT. The blood flows from aorta AT into arteries that deliver the oxygenated blood to the body via the systemic circulatory system.

Blood is additionally received in right atrium RA from coronary sinus CS. Coronary sinus CS collects deoxygenated blood from the heart muscle and delivers it to right atrium RA. Thebesian valve BV is a semicircular fold of tissue at the opening of coronary sinus CS in right atrium RA. Coronary sinus CS is wrapped around heart H and runs in part along and beneath the floor of left atrium LA right above mitral valve MV, as shown in FIG. 9. Coronary sinus CS has an increasing diameter as it connects to right atrium RA.

Inter-atrial septum IS and fossa ovalis FS are also shown in FIG. 9. Inter-atrial septum IS is the wall that separates right atrium RA from left atrium LA. Fossa ovalis FS is a depression in inter-atrial septum IS in right atrium RA. At birth, a congenital structure called a foramen ovale is positioned in inter-atrial septum IS. The foramen ovale is an opening in inter-atrial septum IS that closes shortly after birth to form fossa ovalis FS. The foramen ovale serves as a functional shunt in utero, allowing blood to move from right atrium RA to left atrium LA to then be circulated through the body. This is necessary in utero, as the lungs are in a sack of fluid and do not oxygenate the blood. Rather, oxygenated blood is received from the mother. The oxygenated blood from the mother flows from the placenta into inferior vena cava IVC through the umbilical vein and the ductus venosus. The oxygenated blood moves through inferior vena cava IVC to right atrium RA. The opening of inferior vena cava IVC in right atrium RA is positioned to direct the oxygenated blood through right atrium RA and the foramen ovale into left atrium LA. Left atrium LA can then pump the oxygenated blood into left ventricle LV, which pumps the oxygenated blood to aorta AT and the systemic circulatory system. This allows the pulmonary circulatory system to be bypassed in utero. Upon birth, respiration expands the lungs, blood begins to circulate through the lungs to be oxygenated, and the foramen ovale closes to form fossa ovalis FS.

When patients have abnormal heart function, cardiovascular implants like shunts, stents, and docking devices (for example, devices 300, 400, 500, 600, 700, and 800 shown in FIGS. 10A-19) can help a patient have improved heart function. Cardiovascular implants are small and are typically made of a superelastic or shape-memory material that can have a low radiopacity. The combination of small size and low radiopacity makes visualizing the cardiovascular device within the heart during implantation difficult.

FIGS. 10A-19 below show different examples of cardiovascular implants that can be made with materials and methods described in FIGS. 1-7. FIGS. 20A-20B below show different examples of delivery system components that can be made with materials and methods described in FIGS. 1-7.

### Shunt Device 300

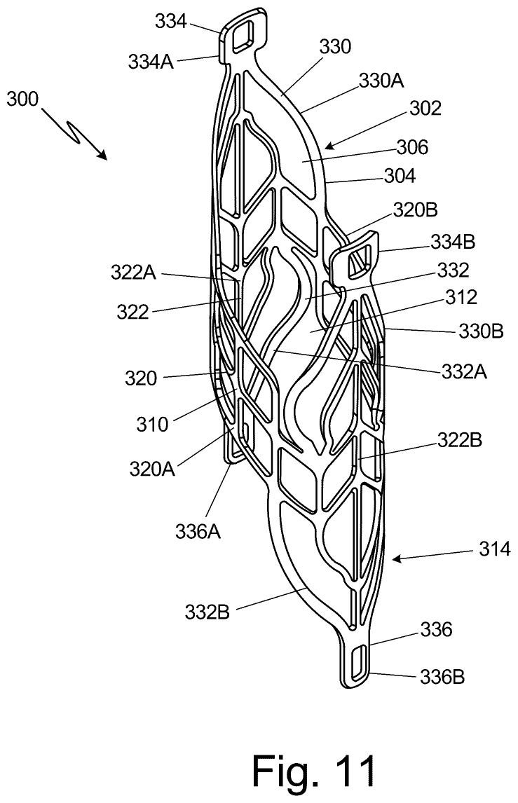

FIG. 10A is a perspective view of shunt device 300. FIG. 10B is a side view of shunt device 300. FIG. 10C is a bottom view of shunt device 300. FIG. 11 is a perspective view of shunt device 300 in a collapsed configuration. FIGS. 10A, 10B, 10C, and 11 will be discussed together. Shunt device 300 includes body 302, which is formed of struts 304 and openings 306. Body 302 includes central flow tube 310, flow path 312, and arms 314. Central flow tube 310 has side walls 320 (including side wall 320A and side wall 320B) and end walls 322 (including end wall 322A and end wall 322B). Arms 314 include distal arms 330 (including distal arm 330A and distal arm 330B) and proximal arms 332 (including proximal arm 332A and proximal arm 332B). Distal arms 330 have terminal ends 334 (including terminal end 331A and terminal end 331B). Proximal arms 332 have terminal ends 336 (including terminal end 336A and terminal end 336B). FIG. 10B further shows horizontal reference plane HP, end wall axis EA, and angle α. FIG. 10C further shows vertical reference plane VP.

Shunt device 300 is shown in an expanded configuration in FIGS. 10A-10C. Shunt device 300 is formed of a super-elastic material, for example a nickel-titanium-hafnium-aluminum alloy as described in FIG. 1 that is capable of being compressed into a catheter for delivery into the body. Shunt device 300 is shown in a compressed configuration in FIG. 11. Upon delivery into the body, shunt device 300 will expand back to its relaxed, or expanded, shape. Shunt device 300 has body 302 that is formed of interconnected struts 304. Openings 306 in body 302 are defined by struts 304. Body 302 of shunt device 300 is formed of struts 304 to increase the flexibility of shunt device 300 to enable it to be compressed and expanded. Shunt device 300 can be sterilized before being delivered into the body.

Body 302 includes central flow tube 310 that forms a center portion of shunt device 300. Central flow tube 310 is tubular in cross-section but is formed of struts 304 and openings 306. Central flow tube 310 can be positioned in a puncture in a tissue wall and holds the tissue wall open. Flow path 312 is an opening extending through central flow tube 310. Flow path 312 is the path through which blood flows through shunt device 300. Arms 314 extend from central flow tube 310. Arms 314 extend outward from central flow tube 310 when shunt device 300 is in an expanded configuration. Arms 314 hold shunt device 300 in position in the tissue wall when shunt device 300 is implanted in the body.

When shunt device 300 is implanted in the tissue wall between the left atrium and the coronary sinus, central flow tube 310 holds the tissue wall open so blood can flow from the left atrium to the coronary sinus through flow path 312. Struts 304 of central flow tube 310 form a cage of sorts that is sufficient to hold the tissue wall open around central flow tube 310. Central flow tube 310 is designed to have a thickness that approximates the thickness of the tissue wall between the left atrium and the coronary sinus.

Central flow tube 310 has side walls 320 and end walls 322. Side wall 320A and side wall 320B form opposing sides of central flow tube 310. End wall 322A and end wall 322B form opposing ends of central flow tube 310. End wall 322A and end wall 322B each extend between and connect to side wall 320A and side wall 320B to form a circular opening that defines flow path 312. Struts 304 of central flow tube 310 define generally parallelogram-shaped openings 306 in central flow tube 310. Struts 304 of side walls 320 form an array of parallelogram-shaped openings 306 (or cells) in the side walls 320. Side walls 320 and end walls 322 form a tubular lattice for creating the central flow tube 310.

As shown in FIG. 10B, central flow tube 310 is preferably angled with respect to horizontal reference plane HP extending through shunt device 300. Horizontal reference plane HP lies generally in the plane of the tissue wall immediately adjacent to shunt device 300 when shunt device 300 is implanted. End walls 322 are angled with respect to horizontal reference plane HP. As shown in FIG. 10B, end walls 322 extend along end wall axis EA that extends at angle α with respect to horizontal reference plane HP. Angle α can be between 15° and 90°. Alternatively, angle α can be between 30° and 75°. Alternatively, angle α can be between 60° and 65°.

Arms 314 of shunt device 300 include two distal arms 330 and two proximal arms 332. Arms 314 extend outward from end walls 322 of central flow tube 310 when shunt device 300 is in an expanded configuration. Distal arm 330A is connected to and extends away from end wall 322A, and distal arm 330B is connected to and extends away from end wall 322B. Proximal arm 332A is connected to and extends away from end wall 322A, and proximal arm 332B is connected to and extends away from end wall 322B. When shunt device 300 is implanted in the tissue wall between the left atrium and the coronary sinus, distal arms 330 will be positioned in the left atrium and proximal arms 332 will be positioned in the coronary sinus.

Distal arms 330 and proximal arms 332 curl outward from end walls 322. As shown in FIG. 10C, distal arm 330A and distal arm 330B extend outwards from central flow tube 310 in opposite directions parallel to vertical reference plane VP. Distal arm 330A has a longer length than distal arm 330B. Proximal arm 332A and proximal arm 332B extend outwards from central flow tube 310 in opposite directions parallel to vertical reference plane VP. Proximal arm 332A has a shorter length than proximal arm 332B. Distal arm 330A has generally the same length and shape as proximal arm 332B, and distal arm 330B has generally the same length and shape as proximal arm 332A. As such, shunt device 300 is inversely symmetrical across horizontal reference plane HP, as shown in FIG. 10B.

Shunt device 300 is generally elongated longitudinally but is relatively narrow laterally. Stated another way, distal arms 330 and proximal arms 332 are not annular or circular but rather extend outward generally in only one plane. As shown in FIG. 10B, shunt device 300 has a general H-shape when viewing a side of shunt device 300. The elongated shape of shunt device 300 means that when compressed it elongates along a line, as shown in FIG. 11, so as to better fit within a catheter.

Distal arms 330 each have terminal ends 334. Specifically, distal arm 330A has terminal end 331A, and distal end 330B has terminal end 331B. Proximal arms 332 each have terminal ends 336. Specifically, proximal arm 332A has terminal end 336A, and proximal arm 332B has terminal end 336B. Terminal ends 334 of distal arms 330 and terminal ends 336 of proximal arms 332 converge towards one another. Distal arms 330 and proximal arms 332 form two pairs of arms. Distal arm 330A and proximal arm 332A form a first pair of arms extending outward from a first side of central flow tube 310, and terminal end 339A of distal arm 330A converges towards terminal end 136A of proximal arm 332A. Distal arm 330B and proximal arm 332B form a second pair of arms extending outward from a second side of central flow tube 310, and terminal end 339B of distal arm 330B converges towards terminal end 336B of proximal arm 332B. The gap between terminal ends 334 and terminal ends 336 is sized to be slightly smaller than an approximate thickness of the tissue wall between the left atrium and the coronary sinus. This allows distal arms 330 and proximal arms 332 to flex outwards and grip the tissue wall when implanted to help hold shunt device 300 in place in the tissue wall. Terminal ends 334 of distal arms 330 and terminal ends 336 of proximal arms 332 can also have openings that are configured to engage a delivery tool to facilitate implantation of shunt device 300, for example actuating rods of a delivery tool.

When implanted in the tissue wall, distal arms 330 and proximal arms 332 are designed such that the projection of distal arms 330 and proximal arms 332 into the left atrium and the coronary sinus, respectively, is minimized. This minimizes the disruption of the natural flow patterns in the left atrium and the coronary sinus. Shunt device 300 can also be designed so that the profile of proximal arms 332 projecting into the coronary sinus is lower than the profile of distal arms 330 projecting into the left atrium to minimize disruption of the natural blood flow through the coronary sinus.

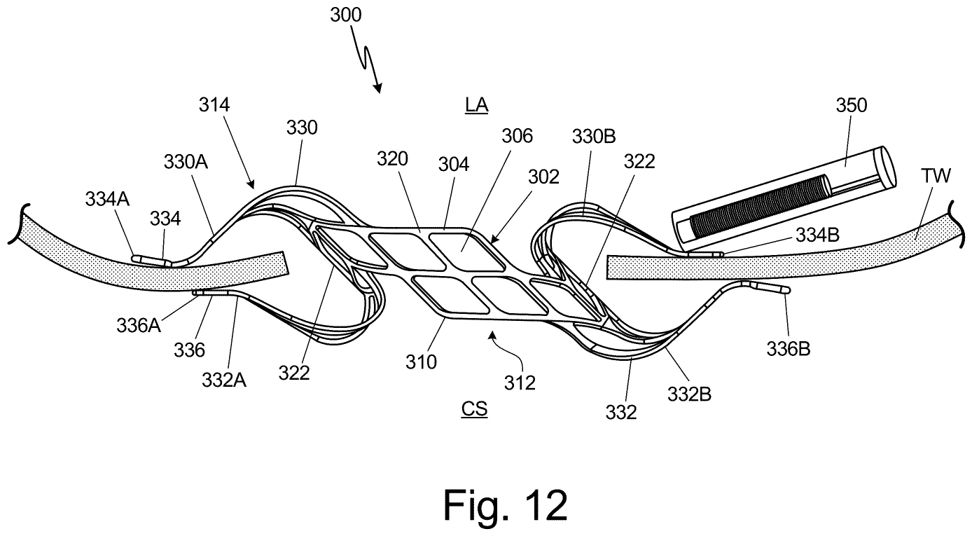

FIG. 12 is a side view of shunt device 300 with sensor 350 and anchored to tissue wall TW. Shunt device 300 includes body 302, which is formed of struts 304 and openings 306. Body 302 includes central flow tube 310, flow path 312, and arms 314. Central flow tube 310 has side walls 320 and end walls 322. Arms 314 include distal arms 330 (including distal arm 330A and distal arm 330B) and proximal arms 332 (including proximal arm 332A and proximal arm 332B). Distal arms 330 have terminal ends 334 (including terminal end 331A and terminal end 331B). Proximal arms 332 have terminal ends 336 (including terminal end 336A and terminal end 336B). FIG. 12 further shows sensor 350, tissue wall TW, left atrium LA, and coronary sinus CS.

Shunt device 300 is described above in reference to FIGS. 10A-12. A variation of the shunt device 300 is shown in FIG. 12, which further includes a sensor 350. Shunt device 300 is shown implanted in tissue wall TW. In the example shown in FIG. 12, sensor 350 will be positioned in left atrium LA when shunt device 300 is implanted in tissue wall TW. Sensor 350 is attached to distal arm 330B of shunt device 300 in the example shown in FIG. 12 but can be attached to distal arm 330A in alternate examples. In further examples, sensor 350 can be attached to proximal arm 332A or proximal arm 332B to be positioned in coronary sinus CS. Alternatively, an additional sensor can be included on shunt device 300 to position a sensor in both left atrium LA and coronary sinus CS. Sensor 350 can be integrally formed with shunt device 300 or attached to shunt device 300 using any suitable mechanism.

Sensor 350 can be a pressure sensor to sense a pressure in the left atrium. In other examples, sensor 350 can be any sensor to measure a parameter in the left atrium. Sensor 350 can include a transducer, control circuitry, and an antenna in one example. The transducer, for example a pressure transducer, is configured to sense a signal from the left atrium. The transducer can communicate the signal to the control circuitry. The control circuitry can process the signal from the transducer or communicate the signal from the transducer to a remote device outside of the body using the antenna. Sensor 350 can include alternate or additional components in other examples. Further, the components of sensor 350 can be held in a sensor housing that is hermetically sealed.

Shunt device 300 can be manufactured at least in part using a wire drawing process as described in FIG. 2A or a tube drawing process as described in FIG. 2B to improve the radiopacity of shunt device 300. Body 302 of shunt device 300 can be manufactured out of a quaternary metal alloy having radiopaque and superelastic properties, similar to alloy 122 and implantable medical device 120 as shown in and discussed in reference to FIGS. 1-7.

### Cardiovascular Implant Device 400

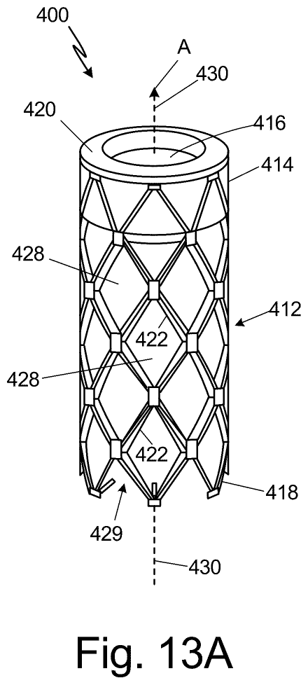

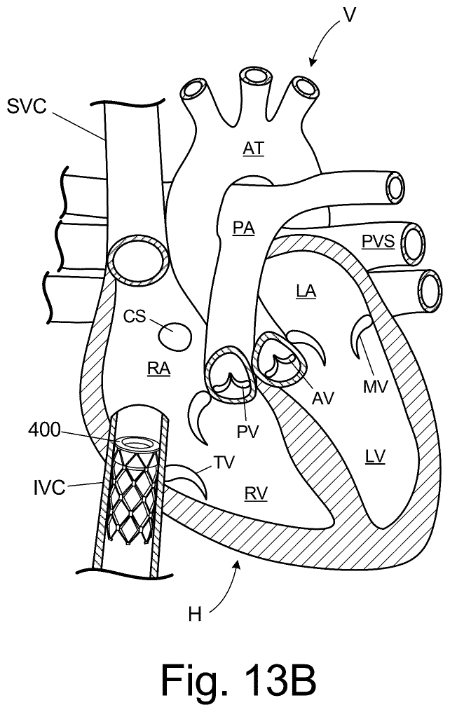

FIG. 13A is a perspective view of cardiovascular implant device 400. FIG. 13B is a sectional view of heart H illustrating an example positioning at a non-valve site of cardiovascular implant device 400.

As illustrated in FIG. 13A, cardiovascular implant device 400 includes frame 412, cover 414, valve seat 416, inflow end 418, and outflow end 420. Frame 412 includes struts 422, inner diameter 424, inner surface 425, outer diameter 426, and outer surface 427, and defines openings 428, central flow path 429, and flow axis 430. FIG. 13B also shows device 400, heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS.

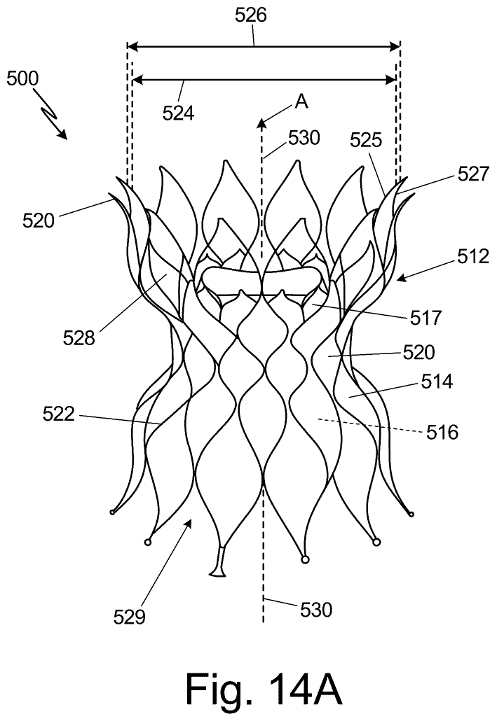

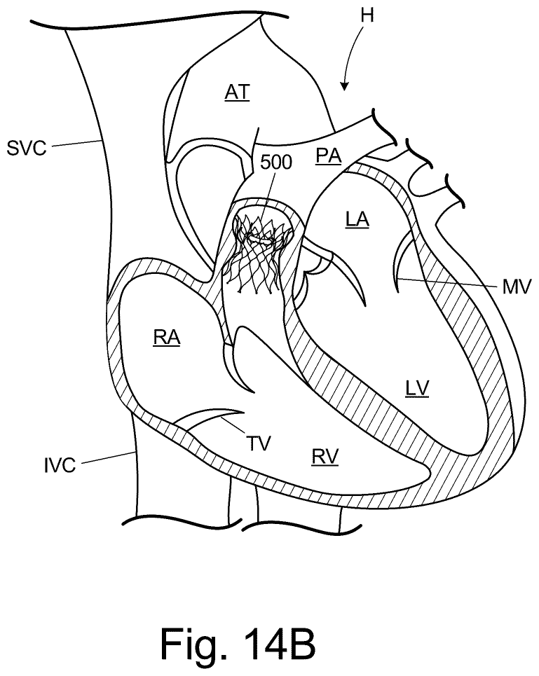

Cardiovascular implant device 400 is an implantable device for use in a cardiovascular system. Cardiovascular implant device 400 is configured to be implanted in blood vessels or chambers of heart H. In the illustrated example, cardiovascular implant device 400 is a “resent” or docking station for supporting a valve device, such as a prosthetic valve device. Cardiovascular implant device 400 can be delivered into the cardiovascular system via a catheter (i.e., transcatheter delivery) or can be surgically placed using transcatheter or surgical procedures known in the art. As shown in FIG. 1B, device 400 is located in inferior vena cava IVC near its opening into right atrium RA. That is, device 400 is located at a site in heart H where there is not naturally a valve (a “non-valve” site). In other examples, device 400 can be located in superior vena cava SVC. In yet other examples, device 400 can be located in any vessel or chamber of heart H at a non-valve site or at a site where there is a natural valve (e.g., aortic valve AV, mitral valve MV, pulmonary valve PV, etc.). Examples of a cardiovascular implant device in a valve site are described below with reference to FIGS. 14A-17.

Frame 412 forms a main body of device 400. Frame 412 can be expandable. Frame 412 can have a wide variety of different shapes and sizes. As shown in FIGS. 13A-13B, frame 412 is an annular or cylindrical mesh or lattice. Frame 412 has inner diameter 424 and outer diameter 426. Each of inner diameter 424 and outer diameter 426 can vary along a length of frame 412. Inner diameter 424 is the diameter of radially inner surface 425 of frame 412. Outer diameter 426 is the diameter of radially outer surface 427 of frame 412. Frame 412 can have any suitable length. For example, frame 412 may be approximately as long as a valve that is configured to sit within frame 412 (e.g., within valve seat 416). In other examples, frame 412 can be longer or shorter than a valve that is configured to sit within frame 412. Frame 412 can press against or into tissue walls at the implant site or contour (or extend) around anatomical structures of the cardiovascular system to set and maintain the position of device 400.

Frame 412 can be formed in a variety of ways, e.g., connecting individual wires together to form a mesh or lattice, braiding, cutting from a sheet and then rolling or otherwise forming into the shape of frame 412, molding, cutting from a cylindrical tube (e.g., cutting from an NiTiHfAl alloy tube), other ways, or a combination of these. Frame 412 can be made from a highly flexible metal, metal alloy, or polymer. Examples of metals and metal alloys that can be used include, but are not limited to, nitinol and other shape-memory alloys such as NiTiHfAl, non-magnetic cobalt-chromium-nickel-molybdenum alloys (Elgiloy), and stainless steel, but other metals and highly resilient or compliant non-metal materials can be used to make frame 412. All or a portion of frame 412 can be monolithically formed of any of these materials. These materials can allow frame 412 to be compressed to a small size, and then—when the compression force is released—frame 412 can self-expand back to its pre-compressed shape. Frame 412 can expand back to its pre-compressed shape due to the material properties frame 412 is made of and/or frame 412 can be expanded by inflation or expansion of a device positioned inside frame 412. For example, frame 412 can be compressed such that frame 412 can fit into a delivery catheter. Frame 412 can also be made of other materials and can be expandable and collapsible in different ways, e.g., mechanically expandable, balloon-expandable, self-expandable, or a combination of these.

Frame 412 extends between inflow end 418 and outflow end 420 of cardiovascular implant device 400. Inflow end 418 can be an end of device 400 that is relatively upstream of outflow end 420 with respect to a flow of blood along flow axis 430, as represented by arrow A in FIG. 13A, when device 400 is implanted in a blood vessel or chamber of heart H. Accordingly, outflow end 420 is an end of device 400 that is relatively downstream of inflow end 418 with respect to a flow of blood along flow axis 430, as represented by arrow A in FIG. 13A, when device 400 is implanted in a blood vessel or chamber of heart H. In the example shown in FIG. 13B, outflow end 420 is positioned near where inferior vena cava IVC opens into right atrium RA and inflow end 418 is positioned upstream within inferior vena cava IVC. Although inflow end 418 is defined as being relatively upstream of outflow end 420, it should be understood that other actual positions of inflow end 418 or outflow end 420 are possible depending on the location where device 400 is implanted.

Frame 412 is formed of a plurality of struts 422. Struts 422 make up the lattice or mesh of frame 412 and define openings (or cells) 428 therein. Struts 422 can be integrally formed. In some examples, all or a portion of struts 422 are monolithically formed from the same material. Openings 428 extend through frame 412 from inner surface 425 to outer surface 427. Each of openings 428 is bounded on one or more sides by one of struts 422. Openings 428 can have any suitable shape or size, which can in turn be based on an overall shape or size of frame 412. In the example shown in FIG. 13A, openings 428 are diamond shaped and arranged in circumferential rows around frame 412. In other examples, openings 428 can have any other regular or irregular polygonal or non-polygonal shape and pattern. In some examples, one of openings 428 can have different shapes or sizes throughout frame 412. In some examples, one of openings 428 can be connected by gaps to adjacent ones of openings 428.

Central flow path 429 is an open channel through a central portion of annular frame 412. Central flow path 429 is defined by inner surface 425 of frame 412. Central flow path 429 extends from inflow end 418 to outflow end 420 such that device 400 is open at each end. Accordingly, blood flowing through and out of device 400 follows central flow path 429. More specifically, flow axis 430 is a longitudinal axis through device 400 along which blood flows as it passes or is directed through device 400 (e.g., in the direction indicated by arrow A in FIG. 13A). In the example illustrated in FIG. 13B where device 400 is implanted in inferior vena cava IVC, a valve seated in device 400 can open when heart H is in a diastolic phase. Blood flows from inferior vena cava IVC and superior vena cava SVC into right atrium RA. The blood that flows from inferior vena cava IVC flows through device 400 along flow axis 430. During the diastolic phase, blood in right atrium RA flows through tricuspid valve TV and into right ventricle RV. During a systolic phase of heart H, a valve seated in device 400 can close. Blood is prevented from flowing (i.e., backflowing) from right atrium RA into inferior vena cava IVC by the closed valve in device 400. A closed valve in device 400 prevents any blood that regurgitates through the tricuspid valve TV during the systolic phase from being forced into inferior vena cava IVC.

Cover 414 is a covering for one or more portions of frame 412. Cover 414 can be a fabric material, a polymer material, or other material. For example, cover 414 can be a material that promotes tissue ingrowth where device 400 contacts adjacent tissue walls of a vessel or chamber of heart H. Cover 414 can also form a seal to limit or prevent blood flow through portions of frame 412 that are covered by cover 414. Cover 414 can be attached to frame 412 by any suitable attachment means, such as by stitching, gluing, tying, etc. Cover 414 can be shaped and positioned in a variety of ways. In the example shown in FIG. 13A, cover 414 is adjacent to outflow end 420. In some examples, cover 414 is near or adjacent valve seat 416. In other examples, cover 414 can be adjacent to inflow end 418 or at any location or locations between inflow end 418 and outflow end 420. In yet other examples, device 400 does not include cover 414.

Valve seat 416 is a portion of device 400 for holding, supporting, or attaching to a valve device, such as a prosthetic valve device. In some examples, valve seat 416 can be a portion of frame 412. In some examples, valve seat 416 can be monolithically formed with frame 412. In other examples, valve seat 416 can be formed separately from frame 412 and attached. Valve seat 416 can take any form that provides a supporting surface for implanting or deploying a valve within device 400 after device 400 is implanted in the cardiovascular system. In the example shown in FIG. 13A, valve seat 416 is located near outflow end 420. However, it should be understood that in other examples valve seat 416 can be located at any lengthwise position along frame 412. Valve seat 416 (and a valve seated therein, not shown) can span across a portion of central flow path 429. Valve seat 416 allows a valve to be implanted in vasculature or tissue of varying strengths, sizes, and shapes. The outer profile of device 400 (e.g., outer surface 427 of frame 412) can better conform to the cardiovascular anatomy (e.g., vasculature, tissue, heart, etc.) without putting too much pressure on the anatomy, while a valve can be firmly and securely implanted in valve seat 416 to prevent or mitigate the risk of migration or slipping.

Once device 400 is implanted in cardiovascular system (e.g., in inferior vena cava IVC as shown in FIG. 13B), circulating blood passes through device 400.

Frame 412 of cardiovascular implant device 400 can be manufactured out of a quaternary metal alloy composed of NiTiHfAl and having radiopaque and superelastic properties, similar to alloy 122 and implantable medical device 120 as shown in and discussed in reference to FIGS. 1-7.

### Cardiovascular Implant Device 500

FIG. 14A is a perspective view of cardiovascular implant device 500. FIG. 14B is a sectional view of heart H illustrating an example positioning at a valve site of cardiovascular implant device 500. FIGS. 14A-14B will be described together. As illustrated in FIGS. 14A-14B, cardiovascular implant device 500 includes frame 512, cover 514, valve seat 516, valve 517, inflow end 518, and outflow end 520. Frame 512 includes struts 522, inner diameter 524, inner surface 525, outer diameter 526, and outer surface 527, and defines openings 528, central flow path 529, and flow axis 530. FIG. 14B also shows device 500, heart H, right atrium RA, right ventricle RV, left atrium LA, left ventricle LV, superior vena cava SVC, inferior vena cava IVC, tricuspid valve TV, pulmonary valve PV, pulmonary artery PA, mitral valve MV, aortic valve AV, and aorta AT.

Cardiovascular implant device 500 includes a similar structure and function to cardiovascular implant device 400 described above, except device 500 is located at a valve site rather than a non-valve site. For example, FIG. 14B shows device 500 is positioned in pulmonary artery PA at the site of pulmonary valve PV such that inflow end 518 is facing right ventricle RV and outflow end 520 is within pulmonary artery PA. In other examples, device 500 can be located at aortic valve AV, mitral valve MV, or other valves. Cardiovascular implant device 500 also includes various minor structural variations compared to device 400. For example, frame 512 has a bi-directionally flared or generally hourglass shaped profile rather than a regular cylindrical shape. Valve seat 516 is located centrally along a longitudinal axis (e.g., flow axis 530) through cardiovascular implant device 500. Cardiovascular implant device 500 is also depicted in FIGS. 14A-14B as including valve 517 supported in valve seat 516. Compared to cover 414 for device 400 as shown in FIG. 13A, cover 514 extends over a greater portion of frame 512. Moreover, cover 514 extends from inflow end 518 towards outflow end 520 but outflow end 520 is not covered by cover 514. It should be understood that, among other variations described above with reference to device 400, other examples of cardiovascular implant devices can include a wide variety of frame shapes and sizes and positions of valve seats and covers.

Once device 500 is implanted in cardiovascular system (e.g., in pulmonary artery PA as shown in FIG. 14B), circulating blood passes through device 500.

Frame 512 of cardiovascular implant device 500 can be manufactured out of a quaternary metal alloy comprised of NiTiHfAl, and having radiopaque and superelastic properties, similar to alloy 122 and implantable medical device 120 as shown in and discussed in reference to FIGS. 1-7.

### Cardiovascular Implant Device 600

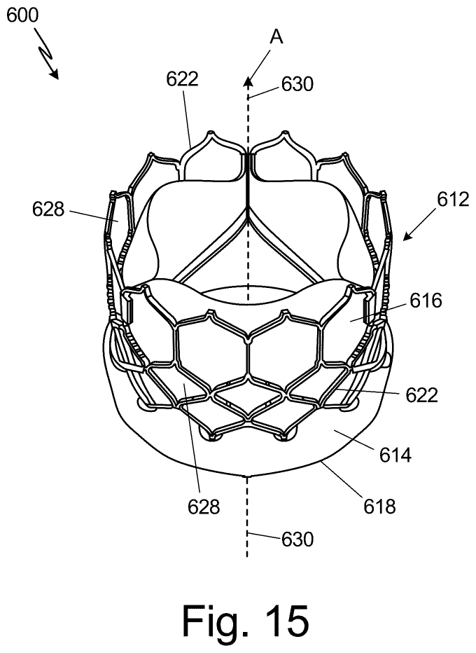

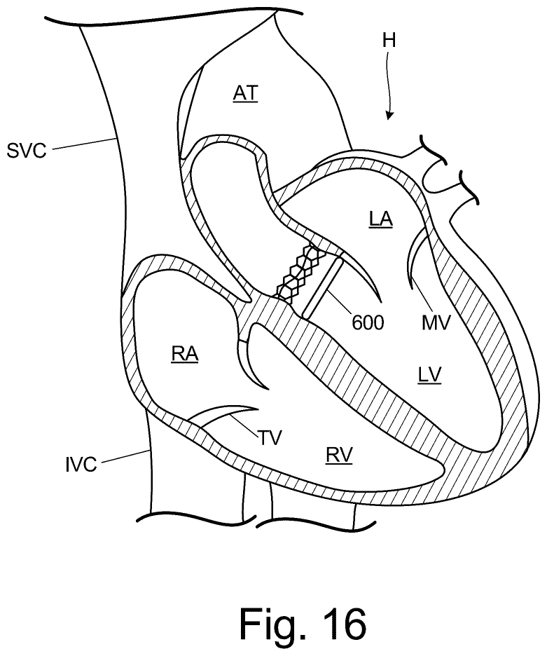

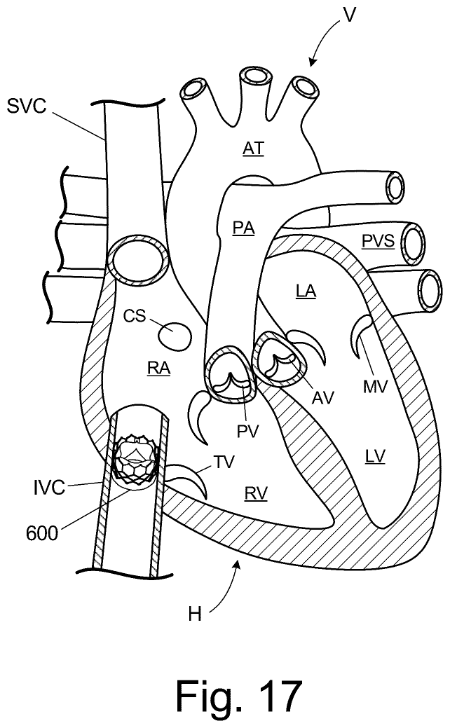

FIG. 15 is a perspective view of cardiovascular implant device 600. FIG. 16 is a sectional view of heart H illustrating an example positioning at a valve site of cardiovascular implant device 600. FIG. 17 is a sectional view of heart H illustrating an example positioning at a non-valve site of cardiovascular implant device 600. FIGS. 15-17 will be described together.

As illustrated in FIG. 15, cardiovascular implant device 600 includes frame 612, cover 614, valvular body 616, inflow end 618, and outflow end 620. Frame 612 includes struts 622, inner diameter 624, inner surface 625, outer diameter 626, and outer surface 627, and defines openings 628, central flow path 629, and flow axis 630. Valvular body includes leaflets 631. FIG. 16 also shows device 600, heart H, right atrium RA, right ventricle RV, left atrium LA, left ventricle LV, superior vena cava SVC, inferior vena cava IVC, tricuspid valve TV, mitral valve MV, aortic valve AV, and aorta AT. FIG. 17 also shows device 600, heart H, vasculature V, right atrium RA, right ventricle RV, left atrium LA, left ventricle LV, superior vena cava SVC, inferior vena cava IVC, tricuspid valve TV, pulmonary valve PV, pulmonary artery PA, pulmonary veins PVS, mitral valve MV, aortic valve AV, aorta AT, and coronary sinus CS.

Cardiovascular implant device 600 is an implantable device for use in a cardiovascular system. Cardiovascular implant device 600 is configured to be implanted in blood vessels or chambers of heart H. In the illustrated example, cardiovascular implant device 600 is a valve device, such as a prosthetic valve device. In some examples, device 600 is deployed into a valve seat of a previously implanted docking station device (e.g., device 400). Cardiovascular implant device 600 can be delivered into the cardiovascular system via a catheter (i.e., transcatheter delivery) or can be surgically placed using transcatheter or surgical procedures known in the art. In some examples, device 600 can be delivered and/or implanted using the same catheter or surgical procedure that is used for a resent device (e.g., device 400). In other examples, device 600 can be delivered and/or implanted by a separate catheter or in a separate surgical procedure. Device 600 can be located in any vessel or chamber of heart H at a site in heart H where there is not naturally a valve (a “non-valve” site) or at a site where there is a natural valve (e.g., aortic valve AV, mitral valve MV, pulmonary valve PV, etc.). For example, FIG. 16 shows an example positioning of device 600 at aortic valve AV (a valve site). In contrast, FIG. 17 shows an example positioning of device 600 in inferior vena cava IVC (a non-valve site).

Frame 612 forms a main body of device 600. Frame 612 can be expandable. Frame 612 can have a wide variety of different shapes and sizes. As shown in FIG. 15, e.g., frame 612 is an annular or cylindrical mesh or lattice. Frame 612 has inner diameter 624 and outer diameter 626. Each of inner diameter 624 and outer diameter 626 can vary along a length of frame 612. Inner diameter 624 is a diameter of radially inner surface 625 of frame 612. Outer diameter 626 is a diameter of radially outer surface 627 of frame 612. Frame 612 can have any suitable length. For example, frame 612 may be approximately as long as valvular body 616. In other examples, frame 612 can be longer than valvular body 616. Frame 612 can press against or into tissue walls at the implant site or contour around anatomical structures of the cardiovascular system to set and maintain the position of device 600.

Frame 612 can be formed in a variety of ways, e.g., connecting individual wires together to form a mesh or lattice, braiding, cutting from a sheet and then rolling or otherwise forming into the shape of frame 612, molding, cutting from a cylindrical tube (e.g., cutting from an NiTiHfAl tube), other ways, or a combination of these. Frame 612 can be made from a highly flexible metal, metal alloy, or polymer. Examples of metals and metal alloys that can be used include, but are not limited to, nitinol and other shape-memory alloys such as NiTiHfAl as discussed above, Elgiloy, and stainless steel, but other metals and highly resilient or compliant non-metal materials can be used to make frame 612. All or a portion of frame 612 can be monolithically formed of any of these materials. These materials can allow frame 612 to be compressed to a small size, and then—when the compression force is released—frame 612 can self-expand back to its pre-compressed shape. Frame 612 can be expanded back to its pre-compressed shape due to the material properties of the material frame 112 is made out of and/or frame 612 can be expanded by inflation or expansion of a device positioned inside the frame. For example, frame 612 can be compressed such that frame 612 can fit into a delivery catheter. Frame 612 can also be made of other materials and can be expandable and collapsible in different ways, e.g., mechanically expandable, balloon-expandable, self-expandable, or a combination of these.

Frame 612 extends between inflow end 618 and outflow end 620 of cardiovascular implant device 600. Inflow end 618 can be an end of device 600 that is relatively upstream of outflow end 620 with respect to a flow of blood along flow axis 630, as represented by arrow A in FIG. 15, when device 600 is implanted in a blood vessel or chamber of heart H. Accordingly, outflow end 620 is an end of device 600 that is relatively downstream of inflow end 618 with respect to a flow of blood along flow axis 630, as represented by arrow A in FIG. 15, when device 600 is implanted in a blood vessel or chamber of heart H. In the example shown in FIG. 16, outflow end 620 is positioned within aorta AT and inflow end 618 is positioned upstream at the site of aortic valve AV (facing left ventricle LV). In the example shown in FIG. 17, outflow end 620 is positioned near where inferior vena cava IVC opens into right atrium RA and inflow end 618 is positioned upstream within inferior vena cava IVC. Although inflow end 618 is defined as being relatively upstream of outflow end 620, it should be understood that other actual positions of inflow end 618 or outflow end 620 are possible depending on the location where device 600 is implanted.

Frame 612 is formed of a plurality of struts 622. Struts 622 make up the lattice or mesh of frame 612 and define openings (or cells) 628 therein. Struts 622 can be integrally formed. In some examples, all or a portion of struts 622 are monolithically formed from the same material comprising an NiTiHfAl alloy. Openings 628 extend through frame 612 from inner surface 625 to outer surface 627. Each of openings 628 is bounded on one or more sides by ones of struts 622. Openings 628 can have any suitable shape or size, which can in turn be based on an overall shape or size of frame 612. In the example shown in FIG. 15, openings 628 are a combination of hexagonal and diamond shaped and arranged in circumferential rows around frame 612. In other examples, openings 628 can have any other regular or irregular polygonal or non-polygonal shape and pattern. In some examples, ones of openings 628 can have different shapes or sizes throughout frame 612. For example, FIG. 15 shows a first row of openings 628 (adjacent outflow end 620) with openings 628 that are hexagonal shaped and the remaining rows of openings 628 with openings 628 that are diamond shaped and smaller. In some examples, ones of openings 628 can be connected by gaps to adjacent ones of openings 628.

Central flow path 629 is an open channel through a central portion of annular frame 612. Central flow path 629 is defined by inner surface 625 of frame 612. Central flow path 629 extends from inflow end 618 to outflow end 620 such that device 600 is open at each end. Accordingly, blood flowing through and out of device 600 follows central flow path 629. More specifically, flow axis 630 is a longitudinal axis through device 600 along which blood flows as it passes or is directed through device 600 (e.g., in the direction indicated by arrow A in FIG. 15). In the example illustrated in FIG. 16 where device 600 is implanted at the site of aortic valve AV, device 600 can be closed (i.e., leaflets 631 of valvular body 616 can close) when heart H is in a diastolic phase. Blood flows from left atrium LA through mitral valve MV into left ventricle LV during the diastolic phase. During a systolic phase of heart H, device 600 can open. Blood flows from left ventricle LV through device 600 along flow axis 630 into aorta AT. In the example illustrated in FIG. 17 where device 600 is implanted in inferior vena cava IVC, device 600 can open when heart H is in a diastolic phase. Blood flows from inferior vena cava IVC and superior vena cava SVC into right atrium RA. The blood that flows from inferior vena cava IVC flows through device 600 along flow axis 630. During the diastolic phase, blood in right atrium RA flows through tricuspid valve TV and into right ventricle RV. During a systolic phase of heart H, device 600 can close. Blood is prevented from flowing (i.e., backflowing) from right atrium RA into inferior vena cava IVC by the closed device 600. A closed device 600 prevents any blood that regurgitates through the tricuspid valve TV during the systolic phase from being forced into inferior vena cava IVC.

Cover 614 is a covering for one or more portions of frame 612. Cover 614 can be a fabric material, a polymer material, or other material. For example, cover 614 can be a material that promotes tissue ingrowth where device 600 contacts adjacent tissue walls of a vessel or chamber of heart H. Cover 614 can also form a seal to limit or prevent blood flow through portions of frame 612 that are covered by cover 614. Cover 614 can be attached to frame 612 by any suitable attachment means, such as by stitching, gluing, tying, etc. Cover 614 can be shaped and positioned in a variety of ways. In the example shown in FIG. 15, cover 614 is adjacent to inflow end 618. In some examples, cover 614 is near or adjacent an attachment region for valvular body 616. In other examples, cover 614 can be adjacent to outflow end 620 or at any location or locations between inflow end 618 and outflow end 620. In yet other examples, device 600 does not include cover 614.

Valvular body 616 is mounted within annular frame 612. More specifically, valvular body 616 is connected to inner surface 625 of frame 612. Valvular body 616 includes one or more leaflets 631. In the example shown in FIG. 15, there are three leaflets 631 (i.e., a tricuspid arrangement). In other examples, valvular body 616 can include more or fewer leaflets 631. The plurality of leaflets 631 are flexible and collapsible within frame 612 to regulate the flow of blood through device 600.

Once device 600 is implanted in cardiovascular system (e.g., in aorta AT at aortic valve AV as shown in FIG. 16 or in inferior vena cava IVC as shown in FIG. 17), circulating blood is delivered through device 600.

Frame 612 of cardiovascular implant device 600 can be manufactured out of a quaternary metal alloy comprising NiTiHfAl and having radiopaque and superelastic properties, similar to alloy 122 and implantable medical device 120 as shown in and discussed in reference to FIGS. 1-7.

### Cardiovascular Implant Device 700

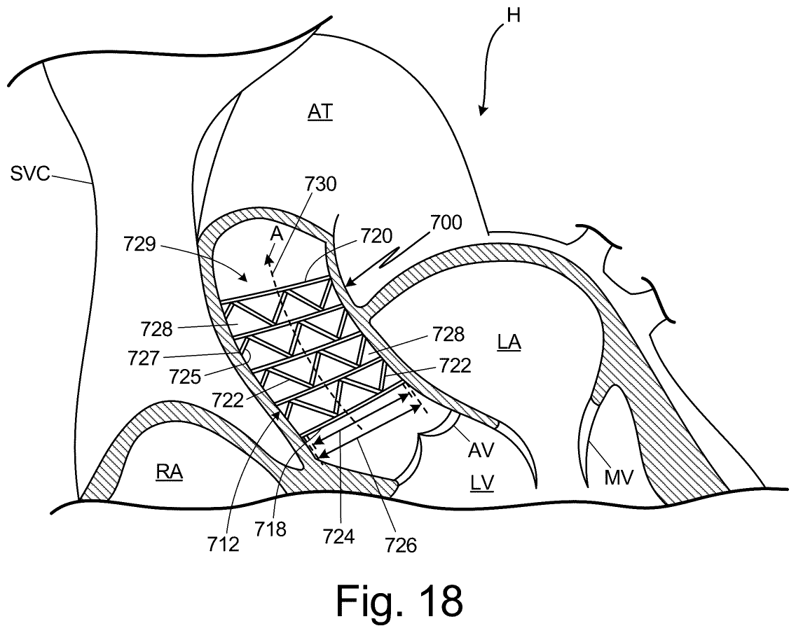

FIG. 18 is a sectional view of heart H illustrating an example positioning of cardiovascular implant device 700. As illustrated in FIG. 18, cardiovascular implant device 700 includes frame 712, inflow end 718, and outflow end 720. Frame 712 includes struts 722, inner diameter 724, inner surface 725, outer diameter 726, and outer surface 727, and defines openings 728, central flow path 729, and flow axis 730. FIG. 18 also shows device 700, heart H, right atrium RA, left atrium LA, left ventricle LV, superior vena cava SVC, mitral valve MV, aortic valve AV, and aorta AT.

Cardiovascular implant device 700 is an implantable device for use in a cardiovascular system. Cardiovascular implant device 700 is configured to be implanted in blood vessels or chambers of heart H. In the illustrated example, cardiovascular implant device 700 is a stent device. Cardiovascular implant device 700 can be delivered into the cardiovascular system via a catheter (i.e., transcatheter delivery) or can be surgically placed using transcatheter or surgical procedures known in the art. In some examples, device 700 can be delivered and/or implanted using the same catheter or surgical procedure that is used for an adjacent (or nearby) resent device (e.g., device 400) or a valve device (e.g., devices 500 and 600). In other examples, device 700 can be delivered and/or implanted by a separate catheter or in a separate surgical procedure. Device 700 can be located in any vessel or chamber of heart H. In some examples, device 700 is located at a site in heart H where there is not naturally a valve (a “non-valve” site). In other examples, device 700 is located near a site where there is a natural valve (e.g., near aortic valve AV, mitral valve MV, pulmonary valve PV, etc.). For example, FIG. 18 shows an example positioning of device 700 within aorta AT.

Frame 712 forms a main body of device 700. Frame 712 can be expandable. Frame 712 can have a wide variety of different shapes and sizes. As shown in FIG. 18, e.g., frame 712 is an annular or cylindrical mesh or lattice. Frame 712 has inner diameter 724 and outer diameter 726. Each of inner diameter 724 and outer diameter 726 can vary along a length of frame 712. Inner diameter 724 is a diameter of radially inner surface 725 of frame 712. Outer diameter 726 is a diameter of radially outer surface 727 of frame 712. Frame 712 can have any suitable length. Frame 712 can press against or into tissue walls at the implant site or contour (or extend) around anatomical structures of the cardiovascular system to set and maintain the position of device 700.

Frame 712 can be formed in a variety of ways, e.g., connecting individual wires together to form a mesh or lattice, braiding, cutting from a sheet and then rolling or otherwise forming into the shape of frame 712, molding, cutting from a cylindrical tube (e.g., cutting from an NiTiHfAl tube), other ways, or a combination of these. Frame 712 can be made from a highly flexible metal, metal alloy, or polymer. Examples of metals and metal alloys that can be used include, but are not limited to, nitinol and other shape-memory alloys such as the NiTiHfAl alloys discussed above, Elgiloy, and stainless steel, but other metals and highly resilient or compliant non-metal materials can be used to make frame 712. All or a portion of frame 712 can be monolithically formed of any of these materials. These materials can allow frame 712 to be compressed to a small size, and then-when the compression force is released-frame 712 can self-expand back to its pre-compressed shape. Frame 712 can expand back to its pre-compressed shape due to the material properties of the material frame 712 is made out of and/or frame 712 can be expanded by inflation or expansion of a device positioned inside frame 712. For example, frame 712 can be compressed such that frame 712 can fit into a delivery catheter. Frame 712 can also be made of other materials and can be expandable and collapsible in different ways, e.g., mechanically expandable, balloon-expandable, self-expandable, or a combination of these.

Frame 712 extends between inflow end 718 and outflow end 720 of cardiovascular implant device 700. Inflow end 718 can be an end of device 700 that is relatively upstream of outflow end 720 with respect to a flow of blood along flow axis 730, as represented by arrow A in FIG. 18, when device 700 is implanted in a blood vessel or chamber of heart H. Accordingly, outflow end 720 is an end of device 700 that is relatively downstream of inflow end 718 with respect to a flow of blood along flow axis 730, as represented by arrow A in FIG. 18, when device 700 is implanted in a blood vessel or chamber of heart H. In the example shown in FIG. 18, outflow end 720 is positioned within aorta AT and inflow end 718 is positioned upstream nearer to the site of aortic valve AV. Although inflow end 718 is defined as being relatively upstream of outflow end 720, it should be understood that other actual positions of inflow end 718 or outflow end 720 are possible depending on the location where device 700 is implanted.

Frame 712 is formed of a plurality of struts 722. Struts 722 make up the lattice or mesh of frame 712 and define openings (or cells) 728 therein. Struts 722 can be integrally formed. In some examples, all or a portion of struts 722 are monolithically formed from the same material. Openings 728 extend through frame 712 from inner surface 725 to outer surface 727. Each of openings 728 is bounded on one or more sides by ones of struts 722. Openings 728 can have any suitable shape or size, which can in turn be based on an overall shape or size of frame 712. In the example shown in FIG. 18, openings 728 are triangular and arranged in circumferential rows around frame 712. In other examples, openings 728 can have any other regular or irregular polygonal or non-polygonal shape and pattern. In some examples, ones of openings 728 can have different shapes or sizes throughout frame 712. For example, FIG. 18 shows varying dimensions of openings 728 throughout frame 712. In some examples, ones of openings 728 can be connected by gaps to adjacent ones of openings 728.

Central flow path 729 is an open channel through a central portion of annular frame 712. Central flow path 729 is defined by inner surface 725 of frame 712. Central flow path 729 extends from inflow end 718 to outflow end 720 such that device 700 is open at each end. Accordingly, blood flowing through and out of device 700 follows central flow path 729. More specifically, flow axis 730 is a longitudinal axis through device 700 along which blood flows as it passes or is directed through device 700 (e.g., in the direction indicated by arrow A in FIG. 18). In the example illustrated in FIG. 18 where device 700 is implanted in aorta AT, aortic valve AV (or a prosthetic valve device, such as device 500) opens during a systolic phase of heart H. Blood flows from left ventricle LV through aortic valve AV into aorta AT. Within aorta AT, blood flows through device 700 along flow axis 730.

Although not shown in FIG. 18, device 700 can also include a cover, which can generally include the same structure and function as covers 414 and 514 described above.

Once device 700 is implanted in the cardiovascular system (e.g., in aorta AT as shown in FIG. 18), circulating blood passes through device 700.

Frame 712 of cardiovascular implant device 700 can be manufactured out of a quaternary metal alloy comprising NiTiHfAl, and having radiopaque and superelastic properties, similar to alloy 122 and implantable medical device 120 as shown in and discussed in reference to FIGS. 1-7.

### Cardiovascular Implant Device 800

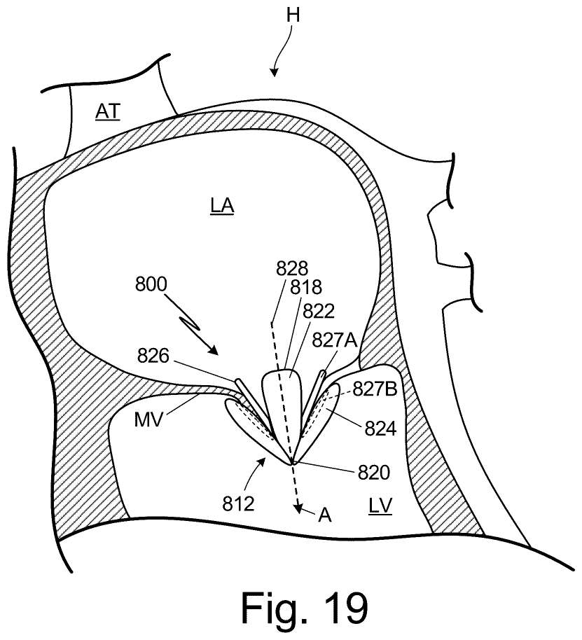

FIG. 19 is a sectional view of heart H illustrating an example positioning of cardiovascular implant device 800. As illustrated in FIG. 19, cardiovascular implant device 800 includes body 812, inflow end 818, and outflow end 820. Body 812 includes central spacer 822, paddles 824, clasps 826 (including first arm 827A and second arm 827B), and central longitudinal axis 828. FIG. 19 also shows device 800, heart H, left atrium LA, left ventricle LV, mitral valve MV, and aorta AT.

Cardiovascular implant device 800 is an implantable device for use in a cardiovascular system. Cardiovascular implant device 800 is configured to be implanted in blood vessels or chambers of heart H. In the illustrated example, cardiovascular implant device 800 is an edge-to-edge valve repair device. Cardiovascular implant device 800 can be delivered into the cardiovascular system via a catheter (i.e., transcatheter delivery) or can be surgically placed using transcatheter or surgical procedures known in the art. In some examples, device 800 can be delivered and/or implanted using the same catheter or surgical procedure that is used for an adjacent (or nearby) stent device (e.g., devices 700 and 800). In other examples, device 800 can be delivered and/or implanted by a separate catheter or in a separate surgical procedure. Device 800 can be located in any vessel or chamber of heart H. In particular, device 800 is located near a site where there is a natural valve (e.g., near mitral valve MV, a tricuspid valve, an aortic valve, a pulmonary valve, etc.). For example, FIG. 19 shows an example positioning of device 800 attached to mitral valve MV. Other examples can include device 800 attached to other natural valves, such as a tricuspid valve, an aortic valve, a pulmonary valve, etc.

Body 812 forms a main body of device 800. Body 812 can be formed in a variety of ways and can be made from a highly flexible metal, metal alloy, or polymer. Examples of metals and metal alloys that can be used include, but are not limited to, nitinol and other shape-memory alloys such as the NiTiHfAl alloys discussed above, Elgiloy, and stainless steel, but other metals and highly resilient or compliant non-metal materials can be used to make body 812. All or a portion of body 812 can be monolithically formed of any of these materials. Body 812, including central spacer 822, paddles 824, and clasps 826, can have an expanded and a closed or collapsed configuration. For example, body 812 can be sized or collapsed to fit into a delivery catheter in a closed configuration. Body 812 can, in some examples, be expanded during an implantation procedure to attach device 800 to a natural valve of heart H.

Body 812 includes central spacer 822. Central spacer 822 forms a central portion of device 800. Central spacer 822 can be generally elongated, cylindrical, or tapered in shape. Central spacer 822 is configured to extend through an opening between leaflets of a natural valve of heart H and maintain a separation between sets of paddles 824 and clasps 826 that bridges the opening between the leaflets. Central longitudinal axis 828 extends longitudinally through central spacer 822.

Clasps 826 are elongated projections from body 812 that extend radially outward from central spacer 822 and central longitudinal axis 828. Clasps 826 include a respective first arm 827A and second arm 827B arranged in a U-shape or V-shape. First arms 827A of clasps 826 are configured to contact or press against a first side of the leaflets of the natural valve of heart H. In the example shown in FIG. 19, first arms 827A contact a side of the leaflets of mitral valve MV that faces left atrium LA. Second arms 827B (shown in dashed lines behind a portion of paddle 824 in FIG. 19) of clasps 826 are configured to contact or press against a second side of the leaflets of the natural valve of heart H. In the example shown in FIG. 19, second arms 827B contact a side of the leaflets of mitral valve MV that faces left ventricle LV. Pairs of first arms 827A and second arms 827B function together to grip the leaflet of leaflets of the natural valve of heart H.

Paddles 824 are paddle shaped or elongated and relatively widened and flattened projections from body 812 that extend radially outward from central spacer 822 and central longitudinal axis 828. Paddles 824 are configured to contact or press against second arms 827B of clasps 826. In the example shown in FIG. 19, paddles 824 are positioned on a side of mitral valve MV that faces left ventricle LV such that paddles 824 are within left ventricle LV. Each of paddles 824 has a corresponding clasp so one of clasps 826 for securing device 800 to the leaflets and holding the leaflets together around device 800. As such, pairs of paddles 824 and corresponding clasps 826 can have complimentary shapes and/or sizes so each pair fits together to grip the leaflet or leaflets. Paddles 824 and clasps 826 can be independently or cooperatively actuated to have different angles with respect to central longitudinal axis 828. An angle of paddles 824 with respect to central longitudinal axis 828 can be adjusted based on the desired amount of contact (i.e., pressure) between paddles 824 and clasps 826 at second arms 827B.

Body 812 extends between inflow end 818 and outflow end 820 of cardiovascular implant device 800. Inflow end 818 can be an end of device 800 that is relatively upstream of outflow end 820 with respect to a flow of blood parallel to central longitudinal axis 828, as represented by arrow A in FIG. 19, when device 800 is implanted in a blood vessel or chamber of heart H. Accordingly, outflow end 820 is an end of device 800 that is relatively downstream of inflow end 818 with respect to a flow of blood parallel to central longitudinal axis 828, as represented by arrow A in FIG. 19, when device 800 is implanted in a blood vessel or chamber of heart H. In the example shown in FIG. 19, outflow end 820 is positioned within left ventricle LV and inflow end 818 is positioned upstream within left atrium LA, such that left atrium LA pumps blood through mitral valve MV, around device 800, and into left ventricle LV. Although inflow end 818 is defined as being relatively upstream of outflow end 820, it should be understood that other actual positions of inflow end 818 or outflow end 820 are possible depending on the location where device 800 is implanted.

Although not shown in FIG. 19, device 800 can also include a cover, which can generally include the same structure and function as covers 314 and 614 described above.

Once device 800 is implanted in the cardiovascular system (e.g., attached to mitral valve MV as shown in FIG. 19), circulating blood passes around device 800.

Body 812 of cardiovascular implant device 800 can also be manufactured out of a quaternary metal alloy comprising NiTiHfAl, and having radiopaque and superelastic properties, similar to alloy 122 and implantable medical device 120 as shown in and discussed in reference to FIGS. 1-7 and the medical devices discussed above.

### Expandable Sheath 900

FIG. 20A is an exploded side view of expandable sheath device assembly 900. FIG. 20B is a side view of expandable sheath device assembly 900 with braided layer 912. FIGS. 20A-20B will be discussed together. FIGS. 20A-B include material from Internation Patent Application No. PCT/US2019/026383, filed Apr. 8, 2019, which claims the benefit of U.S. Application No. 62/722,958, filed Aug. 26, 2019, which claims the benefit of U.S. Application No. 62/655,059, filed Apr. 9, 2018, entitled EXPANDABLE SHEATH, the disclosures of which are hereby incorporated by reference in their entireties.

FIGS. 20A-20B can include expandable sheath device assembly 900, including housing 902 and expandable sheath 904. FIG. 20A further shows introducer 906, dilator 908, and expansion tool 910. FIG. 20B further shows braided layer 912. FIGS. 20A-20B illustrate expandable sheath device assembly 900 (which can be referred to as an introducer device or assembly) that can be used to introduce the medical devices as disclosed above into a patient's body, for example a heart valve, according to one example. Expandable sheath device assembly 900 can comprise housing 902 at a proximal end of expandable sheath device assembly 900 and expandable sheath 904 extending distally from housing 902. Housing 902 can function as a handle for expandable sheath device assembly 900. Expandable sheath 904 has a central lumen (not pictured) to guide passage of the medical device. Generally, during use a distal end of expandable sheath 904 is passed through the skin of the patient and is inserted into a vessel, such as the femoral artery. Any of introducer 906, dilator 908, and expansion tool 910 (shown in FIG. 20A) may be inserted within central lumen of expandable sheath 904, and used to facilitate the insertion and placement of a medical device through expandable sheath 904. For example, expansion tool 910 can be used to pre-expand expandable sheath 904 prior to procedural use. Introducer 906 can be inserted into expandable sheath 904 and a hub of introducer 906 can be releasably connected to housing 902. Dilator 908 can be used to dilate the vessel as necessary to allow for easier insertion of a medical device.

The medical device can then be inserted through housing 902 and expandable sheath 904, and advanced through the patient's vasculature to the treatment site, where the medical device is to be delivered and implanted within the patient. In certain embodiments, housing 902 can include a hemostasis valve that forms a seal around the outer surface of the guide catheter (not pictured) once inserted through the housing to prevent leakage of pressurized blood.

Braided layer 912 can be included wrapped axially around expandable sheath 904. Braided layer 912 can include a plurality of members or filaments braided together. Braided layer 912 can be positioned between an inner layer and an outer layer of expandable sheath 904. The inner layer and the outer layer can be made out of polymeric material in some examples. Braided layer 912 can extend substantially along an entire length of expandable sheath 904 or along only a portion of a length of expandable sheath 904. The filaments of braided layer 912 can be made out of a quaternary metal alloy comprising NiTiHfAl, and having radiopaque and superelastic properties, similar alloy 122 shown in and discussed in reference to FIGS. 1-7 discussed above.

In current devices, sheaths have radiopaque reflectors included at critical junctures of the sheaths, such as at a tip of the sheath. Making braided layer 912 out of a NiTiHfAl alloy as discussed above, more of expandable sheath 904 is visible by using X-ray or other tools to view the radiopaque elements, and the entire length of expandable sheath 904 can be visualized to improve visibility of expandable sheath 904. Braided layer 912 can be composed of any of the alloys disclosed above.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

The treatment techniques, methods, steps, etc. described or suggested herein or in references incorporated herein can be performed on a living animal or on a non-living simulation, such as on a cadaver, cadaver heart, anthropomorphic ghost, simulator (e.g., with the body parts, tissue, etc. being simulated), etc.

### EXEMPLARY ASPECTS

The following are non-exclusive descriptions of possible exemplary aspects of the present invention.

EXAMPLE 1. A device comprising: a shape memory alloy member formed at least partly from a superelastic shape-memory alloy, the alloy displaying reversible stress-induced martensite at a service temperature, such that it has a stress-induced martensitic state and an austenitic state, the shape memory alloy member having (i) a deformed shape when the alloy is in its stress-induced martensitic state and (ii) a different unstressed shape when the alloy is in its austenitic state; wherein the superelastic shape-memory alloy comprises, in atomic percent: a) nickel, b) titanium, c) a first element, wherein the first element is present in an amount effective to impart radiopaque properties to the memory alloy element; and d) a second element wherein the second element is present in an amount effective to balance an optional change in a martensite start temperature Mₛ and an austenite finish temperature A_{f} that occurs due to the presence of the first element and to reach a predetermined transformation temperature; wherein the superelastic shape-memory alloy is thermomechanically processed such that: (i) it exhibits superelastic properties at least at the service temperature, (ii) the alloy is substantially austenitic at zero applied stress at the service temperature, (iii) application of mechanical stress at the service temperature induces a martensitic phase transformation over a strain range, with austenite to martensite transformation stresses in a range of 500-2500 MPa, (iv) removal of the applied stress at the service temperature causes the stress-induced martensite to reversibly transform back to austenite without application of external heating; and wherein the shape memory alloy member exhibits a recoverable strain of 4 to 10% when deformed at the service temperature.

EXAMPLE 2. The device of any one of the examples herein, particularly EXAMPLE 1, wherein the first element is Hf.

EXAMPLE 3. The device of any one of the examples herein, particularly any one of EXAMPLES 1-2, wherein the second element is Al.

EXAMPLE 4. The device of any one of the examples herein, particularly any one of EXAMPLES 1-3, wherein the superelastic shape-memory alloy comprises: a) 49-51 at % of Ni, b) 28-50 at % of Ti; wherein c) the first element is Hf present in an amount of 0.1-20 at %; and d) the second element is Al present in an amount of 0.1-3.5 at %.

EXAMPLE 5. The device of any one of the examples herein, particularly any one of EXAMPLES 1-4, wherein the recoverable strain of the shape memory alloy member is at least 6%.

EXAMPLE 6. The device of any one of the examples herein, particularly any one of EXAMPLES 1-5, wherein the shape memory alloy member exhibits a minimum elongation of 10%, when tested at the service temperature.

EXAMPLE 7. The device of any one of the examples herein, particularly any one of EXAMPLES 1-6, wherein the device is an implantable medical device.

EXAMPLE 8. The device of any one of the examples herein, particularly any one of EXAMPLES 1-7, wherein the service temperature is a body temperature.

EXAMPLE 9. The device of any one of the examples herein, particularly any one of EXAMPLES 1-8, wherein the service temperature is in a range 0 to 40° C.

EXAMPLE 10. The device of any one of the examples herein, particularly any one of EXAMPLES 1-10, wherein the device comprises a shunt, a stent, a valve device, a prosthetic valve device, a docking station, a pre-stent, an edge-to-edge valve repair device, a valve delivery system, a peripheral arterial or coronary stent, a neurovascular stent, a left atrial appendage closure, inferior vena cava filter, a catheter, a surgical instrument, an orthopedic implant, an endovascular aneurysm graft, neurovascular coil, bone anchor or screw, retriever or grasper, intrauterine device (IUD) bone fixation plates, staples, nails, spinal correction rods, bone clips and compression devices, orthodontic archwires, braces, endodontic devices, gastrointestinal tools, and combinations thereof, and wherein the device is configured to induce and remove the stress.

EXAMPLE 11. The device of any one of the examples herein, particularly EXAMPLE 10, wherein the shape memory alloy member comprises a valve frame, a reinforcing member of a sheath, a guide wire, a catheter pull wire, a catheter body, a catheter shaft, or any combination thereof.

EXAMPLE 12. The device of any one of the examples herein, particularly EXAMPLE 11, wherein the reinforcing layer is a braided layer.

EXAMPLE 13. The device of any one of the examples herein, particularly any one of EXAMPLES 1-12, wherein the shape memory alloy member has a thickness in a range of 50 to 250 micrometers.

EXAMPLE 14. The device of any one of the examples herein, particularly any one of EXAMPLES 1-13, wherein the shape memory alloy member exhibits: i) 50-200% increase in radiopacity; and/or ii) 30-400% increase in a strength as compared to a substantially identical reference memory alloy member formed from NiTi alloy.

EXAMPLE 15. A method comprising forming the shape memory alloy member of the device of any one of the examples herein, particularly any one of EXAMPLES 1-14 from an ingot of a superelastic shape-memory alloy comprises, in atomic percent: a) nickel, b) titanium, c) a first element, wherein the first element is present in an amount effective to impart radiopaque properties to the memory alloy element; and d) a second element, wherein the second element is present an amount effective to balance an optional in a martensite start temperature Mₛ and finish A_{f} that occurs due to the presence of Hf and to reach a predetermined transformation temperature.

EXAMPLE 16. The method of claim of any one of the examples herein, particularly EXAMPLE 15, wherein the first element is Hf.

EXAMPLE 17. The method of any one of the examples herein, particularly any one of EXAMPLES 15-16, wherein the second element is Al.

EXAMPLE 18. The method of any one of the examples herein, particularly any one of EXAMPLES 15-17, wherein the superelastic shape-memory alloy comprises: a) 49-51 at % of Ni, b) 28-50 at % of Ti; wherein c) the first element is Hf present in an amount of 0.1-20 at %; and d) the second element is Al present in an amount of 0.1-3.5 at %.

EXAMPLE 19. The method of any one of the examples herein, particularly any one of EXAMPLES 15-18, wherein the ingot is formed by melting the superelastic shape-memory alloy via vacuum induction melting (VIM), vacuum arc melting (VAR), plasma arc melting (PAM), or a combination thereof.

EXAMPLE 20. The method of any one of the examples herein, particularly EXAMPLE 19, wherein the melted superelastic shape memory alloy is homogenized at a temperature of 800-1500° C. for 12 to 96 hours prior to forming the ingot.

EXAMPLE 21. The method of any one of the examples herein, particularly any one of EXAMPLES 15-20, wherein the ingot is formed into a wire, a tube, a sheet, a rod, or any combination thereof.

EXAMPLE 22. The method of any one of the examples herein, particularly EXAMPLE 21, wherein the wire, the tube, the sheet, the rod, or any combination thereof is thermomechanically processed at 400-700° C. prior to forming the memory alloy element.

EXAMPLE 23. The method of any one of the examples herein, particularly any one of EXAMPLES 15-22, wherein the device comprises a shunt, a stent, a valve device, a prosthetic valve device, a docking station, a pre-stent, an edge-to-edge valve repair device, a valve delivery system, a peripheral arterial or coronary stent, a neurovascular stent, a left atrial appendage closure, inferior vena cava filter, a catheter, a surgical instrument, an orthopedic implant, an endovascular aneurysm graft, neurovascular coil, bone anchor or screw, retriever or grasper, intrauterine device (IUD) bone fixation plates, staples, nails, spinal correction rods, bone clips and compression devices, orthodontic archwires, braces, endodontic devices, gastrointestinal tools, and combinations thereof, and wherein the device is configured to induce and remove the stress.

EXAMPLE 24. The method of any one of the examples herein, particularly any one of EXAMPLES 15-23, wherein the shape memory alloy member has a thickness between 50 micrometers and 250 micrometers.

EXAMPLE 25. The method of any one of the examples herein, particularly any one of EXAMPLES 15-24, wherein the shape memory alloy member is surface-treated prior to incorporating it into the device, and wherein the surface treatment comprises surface finishing and/or sterilization.

EXAMPLE 26. The method of any one of the examples herein, particularly EXAMPLE 25, wherein the surface finishing process is chosen from the group consisting of mechanical polishing, chemical etching, chemical polishing, electropolishing, and combinations thereof.

EXAMPLE 27. A method of manufacturing an implantable medical device or a delivery system component for an implantable medical device with radiopaque properties, the method comprising: converting a first ingot of a radiopaque quaternary alloy with composition of Ni₄₉₋₅₁Ti ₂₈₋₃₈Hf₁₀₋₁₉Al _{0.1-3.5} into a radiopaque quaternary alloy form; and forming the implantable medical device or the delivery system component from the radiopaque quaternary alloy form.

EXAMPLE 28. The method of any one of the examples herein, particularly EXAMPLE 27, wherein the implantable medical device comprises: struts; and openings between the struts.

EXAMPLE 29. The method of any one of the examples herein, particularly EXAMPLE 28, wherein the implantable medical device is a cardiovascular implant device chosen from the group consisting of a shunt, a stent, a valve device, a prosthetic valve device, a docking station, a pre-stent, an edge-to-edge valve repair device, and combinations thereof.

EXAMPLE 30. The method of any one of the examples herein, particularly any one of EXAMPLES 27-29, wherein the implantable medical device is between 50 micrometers and 250 micrometers thick.

EXAMPLE 31. The method of any one of the examples herein, particularly any one of EXAMPLES 27-30, wherein the implantable medical device is between 100 micrometers and 180 micrometers thick.

EXAMPLE 32. The method of any one of the examples herein, particularly any one of EXAMPLES 27-31, wherein the radiopaque quaternary alloy form is a laser cut tube, and wherein the laser cut tube is expanded onto a mandrel and heat treated between 450-700 degrees Celsius to impart superelasticity with an upper plateau stress in the range of 800-1100 MPa, a recoverable strain of at least 6%, and a minimum elongation of 10%, when measured using tensile test bars at thirty-seven degrees Celsius.

EXAMPLE 33. The method of any one of the examples herein, particularly any one of EXAMPLES 27-32, wherein forming the implantable medical device or the delivery system component from the radiopaque quaternary alloy form comprises: cutting the implantable medical device or the delivery system component out of the radiopaque quaternary alloy form.

EXAMPLE 34. The method of any one of the examples herein, particularly EXAMPLE 33, wherein cutting the radiopaque quaternary alloy form comprises: laser cutting the implantable medical device or the delivery system component out of the radiopaque quaternary alloy form.

EXAMPLE 35. The method of any one of the examples herein, particularly EXAMPLE 33, and further comprising: shape setting and/or heat treating the implantable medical device or the delivery system component.

EXAMPLE 36. The method of any one of the examples herein, particularly EXAMPLE 33, and further comprising: surface finishing the implantable medical device or the delivery system component using a surface finishing process; and/or sterilizing the implantable medical device or the delivery system component.

EXAMPLE 37. The method of any one of the examples herein, particularly EXAMPLE 36, wherein the surface finishing process is chosen from the group consisting of mechanical polishing, chemical etching, chemical polishing, electropolishing, and combinations thereof.

EXAMPLE 38. The method of any one of the examples herein, particularly any one of EXAMPLES 27-37, wherein converting the first ingot of the radiopaque ternary alloy into a radiopaque quaternary alloy form comprises: melting the first ingot into a slab of radiopaque quaternary alloy; and rolling the slab into a hot worked bar.

EXAMPLE 39. The method of any one of the examples herein, particularly EXAMPLE 38, further comprising drawing the hot worked bar into a wire via cold-working.

EXAMPLE 40. The method of any one of the examples herein, particularly EXAMPLE 39, wherein the wire is heat treated in the range of 475-700 degrees Celsius such that the wire form retains superelasticity with an upper plateau stress in the range of 500-1100 MPa, a recoverable strain of at least 6%, and a minimum elongation of 10%, when measured using tensile testing of straight segments of wire at thirty-seven degrees Celsius.

EXAMPLE 41. The method of any one of the examples herein, particularly any one of EXAMPLES 27-40, wherein the radiopaque quaternary alloy has a composition of Ni₅₀Ti₃₀₋₃₈Hf₁₁₋₁₉Al₁₋₃.

EXAMPLE 42. The method of any one of the examples herein, particularly any one of EXAMPLES 27-41, wherein the radiopaque quaternary alloy has a composition of Ni₅₀Ti₃₆Hf₁₂Al₂.

EXAMPLE 43. The method of any one of the examples herein, particularly any one of EXAMPLES 27-42, wherein the radiopaque quaternary alloy has a composition of Ni₅₀Ti₃₃Hf₁₅Al₂.

EXAMPLE 44. The method of any one of the examples herein, particularly any one of EXAMPLES 27-43, wherein the radiopaque quaternary alloy has a composition of Ni₅₀Ti₂₉Hf₁₈Al₃.

EXAMPLE 45. An implantable medical device with radiopaque properties, the implantable medical device comprising: struts; and openings between the struts; wherein the implantable medical device is comprised of a shape-memory alloy with chemical composition of Ni₄₉₋₅₁Ti ₂₈₋₃₈Hf₁₀₋₁₉Al _{0.1-3.5}.

EXAMPLE 46. The implantable medical device of any one of the examples herein, particularly EXAMPLE 45, wherein the radiopaque quaternary alloy has a composition of Ni₅₀Ti₃₀₋₃₈Hf₁₁₋₁₉Al₁₋₃.

EXAMPLE 47. The implantable medical device of any one of the examples herein, particularly any one of EXAMPLES 45-46, wherein the radiopaque quaternary alloy has a composition of Ni₅₀Ti₃₆Hf₁₂Al₂.

EXAMPLE 48. The implantable medical device of any one of the examples herein, particularly any one of EXAMPLES 45-47, wherein the radiopaque quaternary alloy has a composition of Ni₅₀Ti₃₃Hf₁₅Al₂.

EXAMPLE 49. The implantable medical device of any one of the examples herein, particularly any one of EXAMPLES 45-48, wherein the radiopaque quaternary alloy has a composition of Ni₅₀Ti₂₉Hf₁₈Al₃.

EXAMPLE 50. A delivery system component with radiopaque properties, the delivery system component comprising: a braided layer for a sheath device; wherein the braided layer is comprised of a shape-memory alloy with chemical composition of Ni₄₉₋₅₁Ti ₂₈₋₃₈Hf₁₀₋₁₉Al _{0.1-3.5}.

EXAMPLE 51. The delivery system component of any one of the examples herein, particularly EXAMPLE 50, wherein the braided layer includes a plurality of filaments, wherein the plurality of filaments are comprised of a shape-memory alloy with chemical composition of Ni₄₉₋₅₁Ti ₂₈₋₃₈Hf₁₀₋₁₉Al _{0.1-3.5}.

EXAMPLE 52. The delivery system component of any one of the examples herein, particularly any one of EXAMPLES 50-51, wherein the radiopaque quaternary alloy has a composition of Ni₅₀Ti₃₀₋₃₈Hf₁₁₋₁₉Al₁₋₃.

EXAMPLE 53. The delivery system component of any one of the examples herein, particularly any one of EXAMPLES 50-52, wherein the radiopaque quaternary alloy has a composition of Ni₅₀Ti₃₆Hf₁₂Al₂.

EXAMPLE 54. The delivery system component of any one of the examples herein, particularly any one of EXAMPLES 50-53, wherein the radiopaque quaternary alloy has a composition of Ni₅₀Ti₃₃Hf₁₅Al₂.

EXAMPLE 55. The delivery system component of any one of the examples herein, particularly any one of EXAMPLES 50-54, wherein the radiopaque quaternary alloy has a composition of Ni₅₀Ti₂₉Hf₁₈Al₃.

A method of manufacturing an implantable medical device or a delivery system component for an implantable medical device with radiopaque properties can include converting a first ingot of a radiopaque quaternary alloy with a composition of Ni₄₉₋₅₁Ti₂₈₋₃₈Hf₁₀₋₁₉Al_{0.1-3.5} into a radiopaque quaternary alloy form, and forming the implantable medical device or the delivery system component from the radiopaque quaternary alloy form.

The method of the preceding paragraph can optionally include, additionally and/or alternatively, any one or more of the following steps, features, configurations, and/or additional components:

A further embodiment of the method, wherein the implantable medical device includes struts and openings between the struts.

A further embodiment of the method, wherein the implantable medical device is a cardiovascular implant device chosen from the group consisting of a shunt, a stent, a valve device, a prosthetic valve device, a docking station, a pre-stent, an edge-to-edge valve repair device, and combinations thereof.

A further embodiment of the method, wherein the implantable medical device is between 50 micrometers and 250 micrometers thick.

A further embodiment of the method, wherein the implantable medical device is between 100 micrometers and 180 micrometers thick.

A further embodiment of the method, wherein the radiopaque quaternary alloy form is a laser cut tube, and wherein the laser cut tube is expanded onto a mandrel and heat treated between 450-700 degrees Celsius to impart superelasticity with an upper plateau stress in the range of 800-1100 MPa, a recoverable strain of at least 6%, and a minimum elongation of 10%, when measured using tensile test bars at thirty-seven degrees Celsius.

A further embodiment of the method, wherein forming the implantable medical device or the delivery system component from the radiopaque quaternary alloy form comprises:

cutting the implantable medical device or the delivery system component out of the radiopaque quaternary alloy form.

A further embodiment of the method, wherein cutting the radiopaque quaternary alloy form includes laser cutting the implantable medical device or the delivery system component out of the radiopaque quaternary alloy form.

A further embodiment of the method, and further including shape setting and/or heat treating the implantable medical device or the delivery system component.

A further embodiment of the method, and further including surface finishing the implantable medical device or the delivery system component using a surface finishing process, and/or sterilizing the implantable medical device or the delivery system component.

A further embodiment of the method, wherein the surface finishing process is chosen from the group consisting of mechanical polishing, chemical etching, chemical polishing, electropolishing, and combinations thereof.

A further embodiment of the method, wherein converting the first ingot of the radiopaque ternary alloy into a radiopaque quaternary alloy form includes melting the first ingot into a slab of radiopaque quaternary alloy and rolling the slab into a hot worked bar.

A further embodiment of the method, further comprising drawing the hot worked bar into a wire via cold-working.

A further embodiment of the method, wherein the wire is heat treated in the range of 475-700 degrees Celsius such that the wire form retains superelasticity with an upper plateau stress in the range of 500-1100 MPa, a recoverable strain of at least 6%, and a minimum elongation of 10%, when measured using tensile testing of straight segments of wire at thirty-seven degrees Celsius.

A further embodiment of the method, wherein the radiopaque quaternary alloy has a composition of Ni₅₀Ti₃₀₋₃₈Hf₁₁₋₁₉Al₁₋₃.

A further embodiment of the method, wherein the radiopaque quaternary alloy has a composition of Ni₅₀Ti₃₆Hf₁₂Al₂.

A further embodiment of the method, wherein the radiopaque quaternary alloy has a composition of Ni₅₀Ti₃₃Hf₁₅Al₂.

A further embodiment of the method, wherein the radiopaque quaternary alloy has a composition of Ni₅₀Ti₂₉Hf₁₈Al₃.

The above method(s) can be performed on a living animal or on a simulation, such as on a cadaver, cadaver heart, anthropomorphic ghost, or simulator (e.g., with body parts, heart, tissue, etc. being simulated).

In another embodiment, an implantable medical device with radiopaque properties can include struts and openings between the struts, and the implantable medical device can be comprised of a shape-memory alloy with a chemical composition of Ni₄₉₋₅₁Ti₂₈₋₃₈Hf₁₀₋₁₈Al_{0.1-3.5}.

In another example, a delivery system component with radiopaque properties can include a braided layer for a sheath device comprised of a shape-memory alloy with a chemical composition of Ni₄₉₋₅₁Ti₂₈₋₃₈Hf₁₀₋₁₈Al_{0.1-3.5}.

The implantable medical device of the preceding paragraph can optionally include, additionally and/or alternatively, any one or more of the following features, configurations, and/or additional components:

A further embodiment of any of the foregoing implantable medical devices, wherein the radiopaque quaternary alloy has a composition of Ni₅₀Ti₃₀₋₃₈Hf₁₁₋₁₉Al₁₋₃.

A further embodiment of any of the foregoing implantable medical devices, wherein the radiopaque quaternary alloy has a composition of Ni₅₀Ti₃₆Hf₁₂Al₂.

A further embodiment of any of the foregoing implantable medical devices, wherein the radiopaque quaternary alloy has a composition of Ni₅₀Ti₃₃Hf₁₅Al₂.

A further embodiment of any of the foregoing implantable medical devices, wherein the radiopaque quaternary alloy has a composition of Ni₅₀Ti₂₉Hf₁₈Al₃.

In another embodiment, a delivery system component with radiopaque properties can include a braided layer for a sheath device comprised of a shape-memory alloy with a chemical composition of Ni₄₉₋₅₁Ti₂₈₋₃₈Hf₁₀₋₁₉Al_{0.1-3.5} alloy form, and forming the implantable medical device or the delivery system component from the radiopaque quaternary alloy form.

The delivery system component of the preceding paragraph can optionally include, additionally and/or alternatively, any one or more of the following features, configurations, and/or additional components:

A further embodiment of any of the foregoing delivery system components, wherein the braided layer includes a plurality of filaments, wherein the plurality of filaments are comprised of a shape-memory alloy with chemical composition Ni₄₉₋₅₁Ti₂₈₋₃₈Hf₁₀₋₁₉Al_{0.1-3.5}.

A further embodiment of any of the foregoing delivery system components, wherein the radiopaque quaternary alloy has a composition of Ni₄₉₋₅₁Ti₂₈₋₃₈Hf₁₀₋₁₉ₐAl_{0.1-3.5}.

A further embodiment of any of the foregoing delivery system components, wherein the radiopaque quaternary alloy has a composition of Ni₅₀Ti₃₆Hf₁₂Al₂.

A further embodiment of any of the foregoing delivery system components, wherein the radiopaque quaternary alloy has a composition of Ni₅₀Ti₃₃Hf₁₅Al₂.

A further embodiment of any of the foregoing delivery system components, wherein the radiopaque quaternary alloy has a composition of Ni₅₀Ti₂₉Hf₁₈Al₃.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention is not limited to the particular embodiment(s) disclosed, but that the invention will include all embodiments falling within the scope of the appended claims.

1. A device comprising:
a memory shape alloy member formed at least partly from a superelastic shape-memory alloy, the alloy displaying reversible stress-induced martensite at a service temperature such that it has a stress-induced martensitic state and an austenitic state, the shape memory alloy member having (i) a deformed shape when the alloy is in its stress-induced martensitic state and (ii) a different unstressed shape when the alloy is in its austenitic state;
wherein the superelastic shape-memory alloy comprises, in atomic percent:
a) nickel,
b) titanium,
c) a first element, wherein the first element is present in an amount effective to impart radiopaque properties to the memory alloy element; and
d) a second element wherein the second element is present in an amount effective to balance an optional change in a martensite start temperature Mₛ and an austenite finish temperature A_{f} that occurs due to the presence of the first element and to reach a predetermined transformation temperature;
wherein the superelastic shape-memory alloy is thermomechanically processed such that:
i) it exhibits superelastic properties at least at the service temperature (ii) the alloy is substantially austenitic at zero applied stress at the service temperature,
(iii) application of mechanical stress at the service temperature induces a martensitic phase transformation over a strain range, with austenite to martensite transformation stresses in a range of 500-2500 MPa
(iv) removal of the applied stress at the service temperature causes the stress-induced martensite to reversibly transform back to austenite without application of external heating; and
wherein the shape memory alloy member exhibits a recoverable strain of 4 to 10% when deformed at the service temperature.

2. The device of claim 1, wherein the first element is Hf.

3. The device of claim 1, wherein the second element is Al.

4. The device of claim 1, wherein the superelastic shape-memory alloy comprises:
a) 49-51 at % of Ni,
b) 28-50 at % of Ti; wherein
c) the first element is Hf present in an amount of 0.1-20 at %; and
d) the second element is Al present in an amount of 0.1-3.5 at %.

5. The device of claim 1, wherein the recoverable strain of the shape memory alloy member is at least 6%.

6. The device of claim 1, wherein the shape memory alloy member exhibits a minimum elongation of 10%, when tested at the service temperature.

7. The device of claim 1, wherein the device is an implantable medical device.

8. The device of claim 1, wherein the service temperature is a body temperature.

9. The device of claim 1, wherein the service temperature is in a range of 0 to 40° C.

10. The device of claim 1, wherein the device comprises a shunt, a stent, a valve device, a prosthetic valve device, a docking station, a pre-stent, an edge-to-edge valve repair device, a valve delivery system, a peripheral arterial or coronary stent, a neurovascular stent, a left atrial appendage closure, inferior vena cava filter, a catheter, a surgical instrument, an orthopedic implant, an endovascular aneurysm graft, neurovascular coil, bone anchor or screw, retriever or grasper, intrauterine device (IUD) bone fixation plates, staples, nails, spinal correction rods, bone clips and compression devices, orthodontic archwires, braces, endodontic devices, gastrointestinal tools, and combinations thereof, and wherein the device is configured to induce and remove the stress.

11. The device of claim 10, wherein the shape memory alloy member comprises a valve frame, a reinforcing member of a sheath, a guide wire, a catheter pull wire, a catheter body, a catheter shaft, or any combination thereof.

12. The device of claim 11, wherein the reinforcing member is a braided layer.

13. The device of claim 1, wherein the shape memory alloy member has a thickness in a range of 50 to 250 micrometers.

14. The device of claim 1, wherein the shape memory alloy member exhibits:
i) 50-200% increase in radiopacity; and/or
ii) 30-400% increase in a strength,
as compared to a substantially identical reference memory alloy member formed from NiTi alloy.

15. A method comprising
forming the shape memory alloy member of the device of claim 1 from an ingot of a superelastic shape-memory alloy comprises, in atomic percent:
a) nickel,
b) titanium,
c) a first element, wherein the first element is present in an amount effective to impart radiopaque properties to the memory alloy element; and
d) a second element, wherein the second element is present an amount effective to balance an optional change in a martensite start temperature Mₛ and finish A_{f} that occurs due to the presence of Hf and to reach a predetermined transformation temperature.

16. The method of claim 15, wherein the first element is Hf.

17. The method of claim 15, wherein the second element is Al.

18. The method of claim 15, wherein the superelastic shape-memory alloy comprises:
a) 49-51 at % of Ni,
b) 28-50 at % of Ti; wherein
c) the first element is Hf present in an amount of 0.1-20 at %; and
d) the second element is Al present in an amount of 0.1-3.5 at %.

19. The method of claim 15, wherein the ingot is formed by melting the superelastic shape-memory alloy via vacuum induction melting (VIM), vacuum arc melting (VAR), plasma arc melting (PAM), or a combination thereof.

20. The method of claim 19, wherein the melted superelastic shape memory alloy is homogenized at a temperature of 800-1500° C. for 12 to 96 hours prior to forming the ingot.

21. The method of claim 15, wherein the ingot is formed into a wire, a tube, a sheet, a rod, or any combination thereof.

22. The method of claim 21, wherein the wire, the tube, the sheet, the rod, or any combination thereof is thermomechanically processed at 400-700° C. prior to forming the memory alloy element.

23. The method of claim 15, wherein the device comprises a shunt, a stent, a valve device, a prosthetic valve device, a docking station, a pre-stent, an edge-to-edge valve repair device, a valve delivery system, a peripheral arterial or coronary stent, a neurovascular stent, a left atrial appendage closure, inferior vena cava filter, a catheter, a surgical instrument, an orthopedic implant, an endovascular aneurysm graft, neurovascular coil, bone anchor or screw, retriever or grasper, intrauterine device (IUD) bone fixation plates, staples, nails, spinal correction rods, bone clips and compression devices, orthodontic archwires, braces, endodontic devices, gastrointestinal tools, and combinations thereof, and wherein the device is configured to induce and remove the stress.

24. The method of claim 15, wherein the shape memory alloy member has a thickness between 50 micrometers and 250 micrometers.

25. The method of claim 15, wherein the shape memory alloy member is surface-treated prior to incorporating it into the device, and wherein the surface treatment comprises surface finishing and/or sterilization.

26. The method of claim 25, wherein the surface finishing process is chosen from the group consisting of mechanical polishing, chemical etching, chemical polishing, electropolishing, and combinations thereof.
